SEQUENCE LISTING (4 sequences)

SEQ ID NO: 1 - protein, 183 aa, Homo sapiens
MTTASTSQVRQNYHQDSEAAINRQINLELYASYVYLSMSYYFDRDDVALKNFAKYFLHQSHEEREHAEKLMKLQNQRGGRIFLQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNES

SEQ ID NO: 2 - DNA, 591 bp, Homo sapiens
atggctgatatcggatccatacatatgacgaccgcgtccacctcgcaggtgcgccagaactaccaccaggactcagaggccgccatcaaccgccagatcaacctggagctctacgcctcctacgtttacctgtccatgtcttactactttgaccgcgatgatgtggctttgaagaactttgccaaatactttcttcaccaatctcatgaggagagggaacatgctgagaaactgatgaagctgcagaaccaacgaggtggccgaatcttccttcaggatatcaagaaaccagactgtgatgactgggagagcgggctgaatgcaatggagtgtgcattacatttggaaaaaaatgtgaatcagtcactactggaactgcacaaactggccactgacaaaaatgacccccatttgtgtgacttcattgagacacattacctgaatgagcaggtgaaagccatcaaagaattgggtgaccacgtgaccaacttgcgcaagatgggagcgcccgaatctggcttggcggaatatctctttgacaagcacaccctgggagacagtgataatgaaagctaacctaggcacctcgag

SEQ ID NO: 3 - DNA, 3216 bp, Homo sapiens
ttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccagaacttagtttcgacggattctagaactagtggatcccccatcacaagtttgtacaaaaaagcaggctttaaaggaaccaattcagtcgactggatccatacatatgacgaccgcgtccacctcgcaggtgcgccagaactaccaccaggactcagaggccgccatcaaccgccagatcaacctggagctctacgcctcctacgtttacctgtccatgtcttactactttgaccgcgatgatgtggctttgaagaactttgccaaatactttcttcaccaatctcatgaggagagggaacatgctgagaaactgatgaagctgcagaaccaacgaggtggccgaatcttccttcaggatatcaagaaaccagactgtgatgactgggagagcgggctgaatgcaatggagtgtgcattacatttggaaaaaaatgtgaatcagtcactactggaactgcacaaactggccactgacaaaaatgacccccatttgtgtgacttcattgagacacattacctgaatgagcaggtgaaagccatcaaagaattgggtgaccacgtgaccaacttgcgcaagatgggagcgcccgaatctggcttggcggaatatctctttgacaagcacaccctgggagacagtgataatgaaagctaacctaggcacctcgagatatctagacccagctttcttgtacaaagtggtgatgggctgcaggaattcgatatcaagcttatcgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccgcataggccactagtggatctgatatcacctaataacttcgtatagcatacattatacgaagttatattaagggttctcgagagctcgttttatttaggttctatcgaggagaaaaagcgacaagaagagatagaccatggataaatgattatgttctaaacactcctcagaagctcatcgaactgtcatcctgcgtgaagattaaaatccaacttagaaatttcgagcttacggagacaatcatatgggagaagcaattggaagatagaaaaaaggtactcggtacataaatatatgtgattctgggtagaagatcggtctgcattggatggtggtaacgcatttttttacacacattacttgcctcgagcatcaaatggtggttattcgtggatctatatcacgtgatttgcttaagaattgtcgttcatggtgacacttttagctttgacatgattaagctcatctcaattgatgttatctaaagtcatttcaactatctaagatgtggttgtgattgggccattttgtgaaagccagtacgccagcgtcaatacactcccgtcaattagttgcaccatgtccacaaaatcatataccagtagagctgagactcatgcaagtccggttgcatcgaaacttttacgtttaatggatgaaaagaagaccaatttgtgtgcttctcttgacgttcgttcgactgatgagctattgaaacttgttgaaacgttgggtccatacatttgccttttgaaaacacacgttgatatcttggatgatttcagttatgagggtactgtcgttccattgaaagcattggcagagaaatacaagttcttgatatttgaggacagaaaattcgccgatatcggtaacacagtcaaattacaatatacatcgggcgtttaccgtatcgcagaatggtctgatatcaccaacgcccacggggttactggtgctggtattgttgctggcttgaaacaaggtgcgcaagaggtcaccaaagaaccaaggggattattgatgcttgctgaattgtcttccaagggttctctagcacacggtgaatatactaagggtaccgttgatattgcaaagagtgataaagatttcgttattgggttcattgctcagaacgatatgggaggaagagaagaagggtttgattggctaatcatgaccccaggtgtaggtttagacgacaaaggcgatgcattgggtcagcagtacagaaccgtcgacgaagttgtaagtggtggatcagatatcatcattgttggcagaggacttttcgccaagggtagagatcctaaggttgaaggtgaaagatacagaaatgctggatgggaagcgtaccaaaagagaatcagcgctccccattaattatacaggaaacttaatagaacaaatcacatatttaatctaatagccacctgcattggcacggtgcaacactacttcaacttcatcttacaaaaagatcacgtgatctgttgtattgggatctctagacctaataacttcgtatagcatacattatacgaagttatattaagggttgtcgacctg

SEQ ID NO: 4 - DNA, 4379 bp, Homo sapiens
cggtcaagaagacatatttgacctcttaacaggttcagacgcgactgcctcatcagtaagacccgttgaaaagaacttacctgaaaaaaacgaatatatactagcgttgaatgttagcgtcaacaacaagaagtttaatgacgcggaggccaaggcaaaaagattccttgattacgtaagggagttagaatcattttgaataaaaaacacgctttttcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccagaacttagtttcgacggattctagaactagtggatcccccatcacaagtttgtacaaaaaagcaggctttaaaggaaccaattcagtcgactggatccatacatatgacgaccgcgtccacctcgcaggtgcgccagaactaccaccaggactcagaggccgccatcaaccgccagatcaacctggagctctacgcctcctacgtttacctgtccatgtcttactactttgaccgcgatgatgtggctttgaagaactttgccaaatactttcttcaccaatctcatgaggagagggaacatgctgagaaactgatgaagctgcagaaccaacgaggtggccgaatcttccttcaggatatcaagaaaccagactgtgatgactgggagagcgggctgaatgcaatggagtgtgcattacatttggaaaaaaatgtgaatcagtcactactggaactgcacaaactggccactgacaaaaatgacccccatttgtgtgacttcattgagacacattacctgaatgagcaggtgaaagccatcaaagaattgggtgaccacgtgaccaacttgcgcaagatgggagcgcccgaatctggcttggcggaatatctctttgacaagcacaccctgggagacagtgataatgaaagctaacctaggcacctcgagatatctagacccagctttcttgtacaaagtggtgatgggctgcaggaattcgatatcaagcttatcgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccgcataggccactagtggatctgatatcacctaataacttcgtatagcatacattatacgaagttatattaagggttctcgagagctcgttttatttaggttctatcgaggagaaaaagcgacaagaagagatagaccatggataaatgattatgttctaaacactcctcagaagctcatcgaactgtcatcctgcgtgaagattaaaatccaacttagaaatttcgagcttacggagacaatcatatgggagaagcaattggaagatagaaaaaaggtactcggtacataaatatatgtgattctgggtagaagatcggtctgcattggatggtggtaacgcatttttttacacacattacttgcctcgagcatcaaatggtggttattcgtggatctatatcacgtgatttgcttaagaattgtcgttcatggtgacacttttagctttgacatgattaagctcatctcaattgatgttatctaaagtcatttcaactatctaagatgtggttgtgattgggccattttgtgaaagccagtacgccagcgtcaatacactcccgtcaattagttgcaccatgtccacaaaatcatataccagtagagctgagactcatgcaagtccggttgcatcgaaacttttacgtttaatggatgaaaagaagaccaatttgtgtgcttctcttgacgttcgttcgactgatgagctattgaaacttgttgaaacgttgggtccatacatttgccttttgaaaacacacgttgatatcttggatgatttcagttatgagggtactgtcgttccattgaaagcattggcagagaaatacaagttcttgatatttgaggacagaaaattcgccgatatcggtaacacagtcaaattacaatatacatcgggcgtttaccgtatcgcagaatggtctgatatcaccaacgcccacggggttactggtgctggtattgttgctggcttgaaacaaggtgcgcaagaggtcaccaaagaaccaaggggattattgatgcttgctgaattgtcttccaagggttctctagcacacggtgaatatactaagggtaccgttgatattgcaaagagtgataaagatttcgttattgggttcattgctcagaacgatatgggaggaagagaagaagggtttgattggctaatcatgaccccaggtgtaggtttagacgacaaaggcgatgcattgggtcagcagtacagaaccgtcgacgaagttgtaagtggtggatcagatatcatcattgttggcagaggacttttcgccaagggtagagatcctaaggttgaaggtgaaagatacagaaatgctggatgggaagcgtaccaaaagagaatcagcgctccccattaattatacaggaaacttaatagaacaaatcacatatttaatctaatagccacctgcattggcacggtgcaacactacttcaacttcatcttacaaaaagatcacgtgatctgttgtattgggatctctagacctaataacttcgtatagcatacattatacgaagttatattaagggttgtcgacctgtcagatatagtcggattgcccttttaagcaatcaatagtgaaggtcgatattgggaactgtggtggttggcattcgagtttatcattatcaatactcgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatggttagagttgctattaacggtttcggtagaatcggtagattggtcatgagaattgctttgtctagaccaaacgtcgaagttgttgctttgaacgacccattcatcaccaacgactacgctgcttacatgttcaagtacgactccactcacggtagatacgctggtgaagtttcccacgatgacaagcaca

(19) United States
(12) Patent Application Publication
(10) Pub. No.: US 2024/0238335 A1
(43) Pub. Date: Jul. 18, 2024

(54) FERRITIN COMPOSITION TOLERABILITY

(71) Applicant: Sidero Bioscience, LLC, Hummelstown, PA (US)

(72) Inventors: James Connor, Hershey, PA (US); Ralph Keil, Palmyra, PA (US); Darren Wolfe, Cincinnati, OH (US)

(21) Appl. No.: 18/561,528

(22) PCT Filed: May 19, 2022
(86) PCT No.: PCT/US2022/030072

§ 371 Date: Nov. 16, 2023

Related U.S. Application Data
(60) Provisional application No. 63/190,380, filed on May 19, 2021.

(51) Int. Cl. A61K 33/26; A61K 36/064; A61K 38/17; A61P 7/06

(52) U.S. Cl. CPC A61K 33/26; A61K 36/064; A61K 38/1709; A61P 7/06

(57) ABSTRACT

A method comprising administering to a subject a composition comprising a microbe expressing ferritin or (a) a microbe expressing ferritin and (b) elemental iron, wherein the amount of elemental iron in the composition administered (1) to a subject not diagnosed with an iron deficiency disorder can exceed or is in excess of a dosage of elemental iron recommended by a nutritional authority or (2) to a subject diagnosed with an iron deficiency disorder can exceed or is in excess of a dosage of elemental iron recommended by a medical professional without an adverse effect. A method or a method of altering a composition of a gut bacterial microbiome in a subject comprising administering to a subject a composition comprising of a microbe or host expressing ferritin, with the proviso that when a daily dosage of ferritin in the composition is less than 200 milligrams/day, the composition is free of elemental iron.

### TECHNICAL FIELD

The present disclosure relates to the administration of a source of elemental iron and/or ferritin.

### BACKGROUND

Iron is one of life's most important nonorganic substances with major roles in oxygen transport, short-term oxygen storage, and energy generation to name a few of its fundamental roles in organismal physiology. Deficiencies in iron absorption or excesses in iron loss leads to non-optimal blood and/or tissue iron levels with a wide variety of symptoms. Iron deficiency is the most common and widespread nutritional disorder worldwide, affecting up to two billion people.

Current iron replacement options are plainly inadequate and new strategies are desperately needed for persons seeking to maintain an adequate intake of elemental iron as well as persons with body iron deficiency. Intolerance to oral iron supplementation is common and largely due to problematic gastrointestinal (GI) side effects. In addition, many (actually most) individuals with iron deficiency are not responsive to oral iron treatments leaving them iron deficient.

### SUMMARY

Disclosed herein is a method comprising administering to a subject a composition comprising (a) a microbe or host expressing ferritin and (b) elemental iron, wherein the amount of elemental iron in the composition administered (1) to a subject not diagnosed with an iron deficiency disorder can exceed a dosage of elemental iron recommended by a nutritional authority or (2) to a subject diagnosed with an iron deficiency disorder can exceed a dosage of elemental iron recommended by a medical professional without an adverse effect.

Also disclosed herein is a method comprising administering to a subject a composition comprising (a) a microbe or host expressing ferritin and (b) elemental iron, wherein the amount of elemental iron in the composition administered (1) to a subject not diagnosed with an iron deficiency disorder is in excess of a dosage of elemental iron recommended by a nutritional authority or (2) to a subject diagnosed with an iron deficiency disorder is in excess of a dosage of elemental iron recommended by a medical professional.

Further disclosed herein is a method comprising: providing a composition comprising (a) a microbe or host expressing ferritin and (b) elemental iron; and providing instructions for administration of the composition on a dosage basis, wherein the amount of elemental iron in a dosage of the composition (1) to a subject not diagnosed with an iron deficiency disorder exceeds a dosage of elemental iron recommended by a nutritional authority or (2) to a subject diagnosed with an iron deficiency disorder exceeds a dosage of elemental iron recommended by a medical professional.

Still further disclosed herein is a method comprising administering to a subject a composition comprising a microbe or host expressing ferritin, with the proviso that when a daily dosage of ferritin in the composition is less than 200 milligrams/day, the composition is free of elemental iron.

Yet further disclosed herein is a method of altering a composition of a gut bacterial microbiome in a subject comprising, consisting essentially of or consisting of administering to the subject a composition comprising, consisting essentially of or consisting of a microbe or host expressing ferritin, with the proviso that when a daily dosage of ferritin in the composition is less than 200 milligrams/day, the composition is free of elemental iron.

### BRIEF DESCRIPTION OF THE DRAWINGS

FIG. 1 shows the amino acid sequence of human H-ferritin (SEQ ID NO: 1).

FIG. 2 shows the cDNA sequence of human H-ferritin (SEQ ID NO: 2). The start (ATG) and stop (TAA) codons are bolded, and the BamHI (at 5′ end of sequence) and XhoI (at 3′end of sequence) restrictions are underlined.

FIG. 3 is a schematic showing the structure of a ferritin expression cassette containing the human H-ferritin gene, FTH1, and the selectable URA3 gene. The ferritin expression cassette was amplified by PCR from plasmid RLK/pL5659 using oligonucleotide primers 0-730 and 0-731. Double horizontal lines indicate 3.2 kbp ferritin expression cassette; dark arrows indicate locations of primers with arrow indicating direction of DNA synthesis (5′ to 3′); hatched arrows indicate open-reading frames (ORFs) with point of arrow indicating 3′ end of genes (carboxy-terminus of the encoded protein); open arrow indicates the location of the constitute, highly expressed yeast TDH3 promoter with the arrow showing the direction of transcription (5′ to 3′); open block indicates the yeast CYC1 terminator.

FIG. 4 shows the nucleic acid and amino acid sequence of the expression cassette shown in FIG. 3 (SEQ ID NO:3).

FIG. 5 is a schematic illustrating the chromosomal integration of a ferritin expression cassette containing the TDH3 promoter. Double horizontal lines indicate 3.2 kbp ferritin expression cassette; cross-hatched arrows indicate locations of primers with arrow indicating direction of DNA synthesis (5′ to 3′); hatched arrows indicate open-reading frames (ORFs) with point of arrow indicating 3′ end of genes (carboxy-terminus of the encoded protein); open arrow indicates the location of the constitutive, highly expressed yeast TDH3 promoter with the arrow showing the direction of transcription (5′ to 3′); open block indicates the yeast CYC1 terminator.

FIG. 6 shows the nucleic acid sequence of the chromosomally integrated ferritin expression cassette at TDH3 (SEQ ID NO:5) shown in FIG. 5.

FIG. 7 shows the structure of a gene cassette expressing H-ferritin under the control of the yeast TDH3 transcriptional promoter. H-ferritin ORF—open reading frame encoding human H-ferritin; CYC1—transcriptional terminator from the yeast CYC1 gene; and filled rectangles—loxP sites; URA3—yeast (Kluyveromyces lactis) selectable marker; and

FIG. 8 shows a western blot demonstrating the effect of the chromosomal site of integration on the expression of recombinant H-ferritin in transformed yeast. Lane 1—H-ferritin in RLK/P3190; Lanes 2 and 4—H-ferritin in yeast strains having other chromosomal integration sites of the H-ferritin gene; Lane 3—H-ferritin in RLK/P3177, which contains a multicopy, extrachromosomal plasmid; Lane 5—no sample; Lane 6—purified His-tagged recombinant H-ferritin (rH-ferritin); and Lane 7—molecular weight markers.

FIG. 9 compares the hemoglobin recovery in rats treated with a diploid yeast-ferritin complex compared to rats treated with a haploid yeast-ferritin complex in Example 4.

FIG. 10 shows the hematocrit recovery in rats treated with a diploid yeast-ferritin complex compared to rats treated with a haploid yeast-ferritin complex in Example 4.

FIG. 11A shows the iron content of a haploid yeast sample and a diploid yeast sample based on weight percent of a 1 g sample of yeast.

FIG. 11B shows the H-ferritin content of a haploid yeast sample and a diploid yeast sample.

### DETAILED DESCRIPTION

For purposes of the following detailed description, it is to be understood that the invention may assume various alternative variations and step sequences, except where expressly specified to the contrary. Moreover, other than in any operating examples, or where otherwise indicated, all numbers such as those expressing values, amounts, percentages, ranges, subranges and fractions may be read as if prefaced by the word “about.” even if the term does not expressly appear. Accordingly, unless indicated to the contrary, the numerical parameters set forth in the following specification and attached claims are approximations that may vary depending upon the desired results to be obtained by the present invention. At the very least, and not as an attempt to limit the application of the doctrine of equivalents to the scope of the claims, each numerical parameter should at least be construed in light of the number of reported significant digits and by applying ordinary rounding techniques. Where a closed or open-ended numerical range is described herein, all numbers, values, amounts, percentages, subranges and fractions within or encompassed by the numerical range are to be considered as being specifically included in and belonging to the original disclosure of this application as if these numbers, values, amounts, percentages, subranges and fractions had been explicitly written out in their entirety.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard variation found in their respective testing measurements.

As used herein, unless indicated otherwise, a plural term can encompass its singular counterpart and vice versa, unless indicated otherwise. For example, although reference is made herein to “a” species of yeast, a combination (i.e., a plurality) of these components can be used. In addition, in this application, the use of “or” means “and/or” unless specifically stated otherwise, even though “and/or” may be explicitly used in certain instances.

As used herein, “including.” “containing” and like terms are understood in the context of this application to be synonymous with “comprising” and are therefore open-ended and do not exclude the presence of additional undescribed and/or unrecited elements, materials, ingredients and/or method steps.

As used herein, “consisting of” is understood in the context of this application to exclude the presence of any unspecified element, ingredient and/or method step.

As used herein, “consisting essentially of” is understood in the context of this application to include the specified elements, materials, ingredients and/or method steps “and those that do not materially affect the basic and novel characteristic(s)” of what is being described.

As used herein, “patient” or “subject” or “individual” means animals, including mammals, including humans, a canine, a feline, a bovine, an equine, a porcine, a primate, and/or a rodent.

As used herein, “administering” an amount (e.g., a dose) of a composition may be done by the subject himself/herself or another subject (e.g., a medical professional, a caretaker, a family member). The composition may be provided by the subject or the administrator for the subject along with instructions for the administration of the composition (e.g., written instructions on the label of a container containing the composition).

As used herein, “ingestible” means capable of being taken into the body orally.

As used herein, “medical food” means a food that is designed to be consumed or administered enterally for the treatment of a disease that has distinctive nutritional requirements that cannot be met solely by normal diet.

As used herein, “dietary supplement” or “nutritional supplement” means something that is consumed to supplement a subject's diet in addition to meals.

As used herein, “pharmaceutical composition” means any chemical or biological composition, material, agent or the like that is capable of inducing a therapeutic or metabolic effect when properly administered to a subject, including the composition, material, agent or the like in an inactive form and active metabolites thereof, where such active metabolites may be formed in vivo.

As used herein, “composition” means a solution, dispersion, or solid, such as a powder.

As used herein, “iron” refers to elemental iron that may be in the form of a salt such as but not limited to iron oxide, iron sulfate, and iron hydride.

As used herein, “iron deficient” or “iron deficiency” refers to a condition in which subjects have a TSAT of ≤25% and a serum ferritin concentration of ≤100 ng/mL.

As used herein, “anemia” refers to a condition in which subjects have a hemoglobin concentration of less than 13 g/dL; subjects may exhibit symptoms commonly associated with disorders or diseases related to iron deficiency, iron uptake and/or iron metabolism based on responses to a health-related quality of life questionnaire. As used herein, “iron deficiency anemia” refers to a condition in which subjects have a TSAT of less than 20%, a serum ferritin concentration of less than 50 ng/mL, and/or a hemoglobin concentration of less than 13 g/dL. Subjects may exhibit symptoms commonly associated with disorders or diseases related to iron deficiency, iron uptake, and/or iron metabolism based on responses to a health-related quality of life questionnaire. Examples of iron deficiency disorders include iron deficiencies caused by insufficient dietary intake or absorption of iron. Iron deficiency disorders may be related to, for example, malnutrition, pregnancy (including the postpartum period), heavy uterine bleeding, chronic disease (including chronic kidney disease), cancer, renal dialysis, gastric by-pass, multiple sclerosis, restless leg syndrome, diabetes (e.g., Type I or Type II diabetes), insulin resistance, and attention deficit disorders.

As used herein, a “gut microbiome disorder” refers to an imbalance of a patient's or subject's microbiome. In particular, a gut microbiome disorder refers to elevated relative abundance of genus of bacteria that negatively impact the gut microbiome, such as, for example, Klebsiella, Enterobacteriales, Enterobacteriaceae, Clostridium, Anaerosporobacter, and/or Pygmaiobacter.

As used herein, “treat,” “treatment,” or “treating” means a therapeutic, prophylactic or preventative measure provided to a patient or subject with the intention of preventing the development or altering the pathology or symptoms experienced by the patient or subject, such as, those resulting from a disorder, which may include an iron deficiency disorder or a gut microbiome disorder. A “treatment” administered to a patient or subject may achieve any clinically or quantitatively measurable reduction in the condition for which the patient or subject is being treated up to and including complete elimination. “Treatment” refers to both therapeutic treatment and prophylactic or preventative measures. “Treatment” may also be specified as palliative care. “Treatment” may also include administration of the composition as a dietary iron source and/or a ferritin source. Those in need of treatment include those already with one or more iron deficiency disorders as well as those in which the disorder is to be prevented and those who may benefit from supplementation.

As used herein, “dietary management” means treatment of a condition through administration of a medical food, a food ingredient, or a dietary supplement.

As used herein, “recombinant host” means a host, the genome of which is augmented or introduced by at least one recombinant gene. “Augmented” or “introduced” refers to augmentation or introduction caused by human intervention. Exemplary recombinant hosts include but are not limited to plants, fungi such as yeast, bacteria or animals such as porcine.

As used herein, “recombinant gene” is a nucleic acid sequence in which one or more genes or segments of genes have been inserted, resulting in a new genetic combination. A recombinant gene is generally a gene that is not naturally found in the context in which it is used. A recombinant gene may be a gene that is integrated into the chromosome of a recombinant host at an integration site at which the sequence does not naturally occur. The recombinant gene can be a DNA sequence from a species other than the recombinant host. The recombinant gene can be one or more additional copies of a gene that naturally occurs in the recombinant host inserted into additional locations of the recombinant host's genome to allow for overexpression or altered expression of the gene product of that DNA sequence.

As used herein, “haploid” means a cell that contains one set of chromosomes. As used herein, “one set of chromosomes” means the genome of a haploid.

As used herein, “diploid” means a cell that contains two sets of chromosomes that contain pairs of homologous chromosomes.

As used herein “two sets of chromosomes” means the genome of a diploid.

As used herein, “polyploid” means a cell that contains more than two sets of homologous chromosomes.

As used herein, “aneuploid” means a cell that contains an abnormal number of chromosomes. An “abnormal number of chromosomes” means a number of chromosomes that is different than from the usual number of chromosomes in a given organism. For example, an S. cerevisiae cell comprises 16 chromosomes. An aneuploid S. cerevisiae cell may comprise, as examples, 15 chromosomes or 17 chromosomes (i.e., any number that is not 16).

As used herein, “homologous” means pairs of chromosomes of similar centromere position, gene composition and length.

As used herein, “homologue” means one chromosome of a homologous pair.

As used herein, “prototroph” or “prototrophic” means a microorganism that can synthesize its nutrients from inorganic material.

As used herein, “mate”, “mated”, or “mates” is the process whereby two cells of opposite mating types, typically haploids, fuse, generally forming a diploid.

As used herein, “ferritin” is a globular protein that acts as a primary intracellular iron-storage protein in most organisms, including prokaryotes and eukaryotes. Ferritin is a large (nearly 480 kDa) multi-subunit complex comprising 24 polypeptide subunits and is capable of containing as many as 4,500 atoms of iron ions (Fe²⁺ or Fe³⁺) within a hydrous iron oxide core.

As used herein, a “salt” refers to an ionic compound made up of metal cations and non-metallic anions and having an overall electrical charge of zero. Salts may be hydrated or anhydrous.

As used herein, “dry matter,” with respect to a composition of the present invention, means that the composition has no more than 10 percent water by weight based on total weight of the composition. As used herein, “dry matter basis” means a method of expressing the concentration of a component in a composition by expressing the component's concentration in terms of the dry matter content.

As used herein, “organic-iron complexes” are compounds which contain iron in the (II) or (III) oxidation state, complexed with or otherwise bound to (e.g., an ionic bond) an organic compound. Examples of suitable organic-iron complexes include, but are not limited to, iron polymer complexes, iron carbohydrate complexes, and iron aminoglycosan complexes.

As used herein, “closely linked” means a first gene is within five centimorgans of a second gene.

As used herein, “centimorgan” means a unit of measurement that is equal to a one percent chance that a first marker on a chromosome will become separated from a second marker on the same chromosome due to crossing over in a single generation.

As used herein, an “adverse effect” means any untoward medical occurrence in a subject consuming elemental iron or ferritin, and which does not necessarily have a causal relationship with such treatment. An “adverse effect” includes an intake of elemental iron that can cause or lead to issues including gastric upset, constipation, nausea, abdominal pain, vomiting, or faintness. An “adverse effect” also includes an intake of elemental iron that results in hemochromatosis or “iron overload” where excess iron remains in the subject. An “adverse effect” alternatively or additionally means any untoward medical occurrence in a subject consuming ferritin, either in a composition with elemental iron or a composition free of elemental iron, and which does not necessarily have a causal relationship with such treatment. An “adverse effect” includes an intake of ferritin that can cause stomach pain, heart palpitations or chest pains, unexplained weakness or fatigue, or joint pain.

As used herein, “free of elemental iron” or “in the absence of elemental iron” or “in the substantial absence of elemental iron” means that no amount of elemental iron is intentionally added to (exogenously combined with) a microbe or host expressing ferritin in a method of making a composition. It is appreciated that some amount of iron may be present in the microbe or host expressing ferritin (e.g., a yeast may contain some amount of iron). For example, a microbe or host expressing ferritin may be grown in a medium (e.g., a yeast fermentation medium) that may contain trace elements including iron along with vitamins and salts. Such trace elements, vitamins and salts may be present in the microbe or host expressing ferritin.

As used herein, “transferrin saturation” or “TSAT” means the ratio of serum iron to total iron-binding capacity (TIBC).

As used herein, “total iron-binding capacity” or “TIBC” means the total amount of iron that can be bound with serum proteins.

As used herein, “functional selectable marker” or “functional selectable marker gene” means a genetic element that provides for a growth advantage under certain conditions for progeneration of a recombinant host.

As used herein, “growth advantage” means that 90% or more of the progeny of the recombinant host comprise the genetic cassette.

As used herein, “genetic cassette” comprises a functional selectable marker gene and a gene encoding ferritin.

As used herein, “mutant” means a non-functional gene. As used herein, “non-mutant” means a functional gene.

As used herein, a “nutritional authority” is a governmental board, agency, commission, panel or individual that recommends or provides advice on reference values for dosage amounts, e.g., daily dosage amounts for a particular nation, country or state or multiple nations, countries or states. Examples of “nutritional authorities” include, but are not limited to, the Food and Nutrition Board (FNB) at the Institute of Medicine (IOM) of the National Academies (formerly National Academy of Sciences) in the United States; the Panel on Dietetic Products, Nutrition and Allergies of the European Food Safety Authority in Europe; and the Health Service Bureau, Ministry of Health, Labor and Welfare in Japan.

As used herein, a “daily dosage” of elemental iron is an amount of elemental iron consumed in one day, either all at once (one sitting) or through multiple sittings throughout a day. A “daily dosage” may include elemental iron consumed from a single source or multiple different sources.

Disclosed herein is a method comprising, consisting essentially of or consisting of administering to a subject a composition comprising (a) a microbe or host expressing ferritin and (b) elemental iron, wherein the amount of elemental iron in the composition administered (1) to a subject not diagnosed with an iron deficiency disorder can exceed a dosage of elemental iron recommended by a nutritional authority or (2) to a subject diagnosed with an iron deficiency disorder can exceed a dosage of elemental iron recommended by a medical professional without an adverse effect.

Also disclosed herein is a method comprising, consisting essentially of or consisting of administering to a subject a composition comprising (a) a microbe or host expressing ferritin and (b) elemental iron, wherein the amount of elemental iron in the composition administered (1) to a subject not diagnosed with an iron deficiency disorder is in excess of a dosage of elemental iron recommended by a nutritional authority or (2) to a subject diagnosed with an iron deficiency disorder is in excess of a dosage of elemental iron recommended by a medical professional.

Further disclosed herein is a method comprising, consisting essentially of or consisting of: providing a composition comprising (a) a microbe or host expressing ferritin and (b) elemental iron; and providing instructions for administration of the composition on a dosage basis, wherein the amount of elemental iron in a dosage of the composition (1) to a subject not diagnosed with an iron deficiency disorder exceeds a dosage of elemental iron recommended by a nutritional authority or (2) to a subject diagnosed with an iron deficiency disorder exceeds a dosage of elemental iron recommended by a medical professional.

Still further disclosed herein is a method comprising, consisting essentially of or consisting of administering to a subject a composition comprising a microbe or host expressing ferritin, with the proviso that when a daily dosage of ferritin in the composition is less than 200 milligrams/day, the composition is free of elemental iron.

Yet further disclosed herein is a method of altering a composition of a gut bacterial microbiome in a subject comprising, consisting essentially of or consisting of administering to the subject a composition comprising, consisting essentially of or consisting of a microbe or host expressing ferritin, with the proviso that when a daily dosage of ferritin in the composition is less than 200 milligrams/day, the composition is free of elemental iron.

Any nutritional, non-pathogenic, or ingestible microbe or host may be used in the described methods. As used herein. “non-pathogenic” means microbes or hosts that are unable to cause a disease. The microbe or host may be grown specifically for the purpose of iron supplementation, or it may be the product of another process (e.g., fermentation).

Suitable examples of microbes or hosts useful in the invention include, but are not limited to, a fungus, an alga, a bacterium, a protozoan, a virus, microscopic helminths, microorganisms, or combinations thereof. For example, recombinant microbe or host strains suitable for dietary or nutritional supplementation of iron can store iron in a form having high bioavailability for mammals, including humans, such as those that meet the Generally Regarded As Safe (GRAS) requirements for human consumption or New Dietary Ingredient (NDI) requirements or any other regulatory food or food supplement requirement. Other microbes or hosts that can be used in processes to produce dietary or nutritional supplementation of iron also may be used in the composition of the invention. A fungus may be, for example, a yeast. Non-limiting examples of yeast include various species of the genus Saccharomyces, the genus Schizosaccharomyces, the genus Kluyveromyces, or the genus Pichia. Non-limiting examples of algae include various species of the genus Chlamydomonas and non-limiting examples of bacteria include various species of the genus Lactococcus. The microbe or host may contain impurities that may contribute to the weight of a composition of the present invention, but these weights are excluded from the total dry matter weight of the composition.

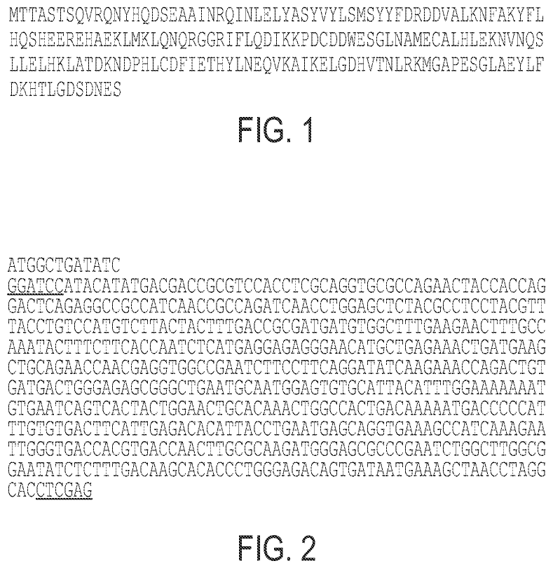

As mentioned above, the microbe or host may express ferritin. For example, the microbe or host may be a recombinant microbe or host including at least one recombinant gene that encodes ferritin or a homologue thereof. The gene that encodes ferritin or a homologue thereof allows the recombinant host to make the ferritin protein through expression of the ferritin gene. Suitable ferritin comprises mammalian or plant H-ferritin and/or L-ferritin subunits. Any H-ferritin subunit from any species whatever may be used, as long as it encodes H-ferritin. The H-ferritin subunits may be mammalian H-ferritin subunits, such as, for example, human, canine, feline, bovine, equine, porcine, primate, and/or rodent. The H-ferritin subunits may be human H-ferritin (FTH1) (SEQ. ID. NO. 1; see FIG. 1; see also FIG. 2). The H-ferritin can also be a naturally occurring or synthetic homologue or variant of human H-ferritin. The H-ferritin homologue may have 80 percent to 100 percent sequence identity to human H-ferritin, such as at least 80 percent, 81 percent, 82 percent, 83 percent, 84 percent, 85 percent, 86 percent, 87 percent, 88 percent, 89 percent, 90 percent, 91 percent, 92 percent, 93 percent, 94 percent, 95 percent, 96 percent, 97 percent, 98 percent, or 99 percent sequence identity with human H-ferritin and retains the ability to bind iron and form a multi-subunit ferritin-iron complex (described below), but can be mutated to provide varying binding and disassociation strengths between the iron and the ferritin. Optionally, the ferritin may be or include L-ferritin. For example, the ferritin subunit may comprise at least 20 percent H-ferritin as compared to L-ferritin, such as about 20 percent, 30 percent, 40 percent, 50 percent, 60 percent, 70 percent, 80 percent, 90 percent, or 100 percent H-ferritin as compared to L-ferritin. Optionally, all of the ferritin subunits (i.e., 100 percent of the ferritin subunits) may be H-ferritin or all of the ferritin subunits (i.e., 100 percent of the ferritin subunits) may be L-ferritin.

According to the invention, the ferritin can be recombinant ferritin. For example, the H-ferritin can be human H-ferritin, or a homologue thereof, produced in a microbial strain comprising a polynucleotide sequence encoding the H-ferritin under the control of an appropriate promoter. The gene encoding ferritin may be extra-chromosomal (episomal) or may be chromosomally integrated.

For example, the microbe or host may be a strain of recombinant microbe or host, such as yeast expressing ferritin from a chromosomally integrated ferritin expression cassette (e.g., a chromosomally integrated H-ferritin expression cassette). In such a case, the ferritin coding sequence may be placed under the control of an appropriate yeast promoter in an iron-storage protein (e.g., H-ferritin) expression cassette to produce high enough levels of the iron-storage protein for the yeast to serve as a suitable vehicle for iron supplementation. Suitable yeast promoters are known in the art and include promoters that induce a high level of constitutive expression or promoters whose expression can be regulated by environmental conditions. In addition, the genetic constitution of the yeast can be further manipulated to achieve a variety of potentially advantageous outcomes. For example, proteolysis may be manipulated to enhance the stability of the iron-storage protein or iron transport mechanisms, including but not limited to those of the cell surface, the vacuole, or the mitochondria, can be manipulated to achieve desirable outcomes such as altering the iron concentration in specific cellular compartments. In addition, the yeast may be altered in other manners to manipulate the level of iron in the iron-storage protein or cellular compartments. The iron content of the yeast may be regulated by adding known amounts of an iron compound to the medium in which the yeast is grown. Using the recombinant yeast, iron supplementation for humans and other animals can be accomplished by any of a number of means including, but not limited to, consumption or ingestion of yeast. The yeast may be grown specifically for the purpose of iron supplementation, or they may be the by-product of another process (e.g., fermentation).

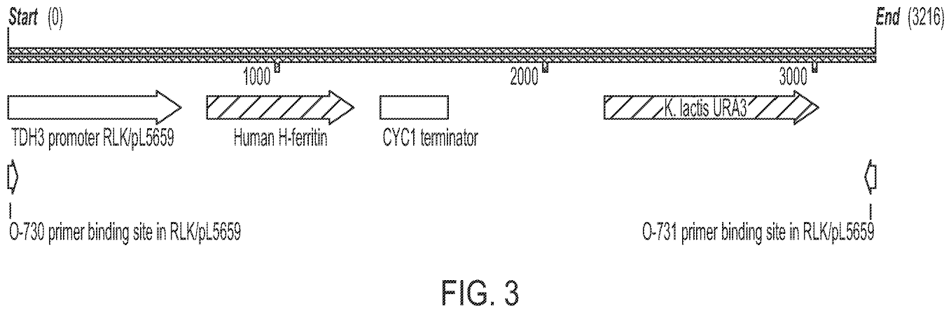

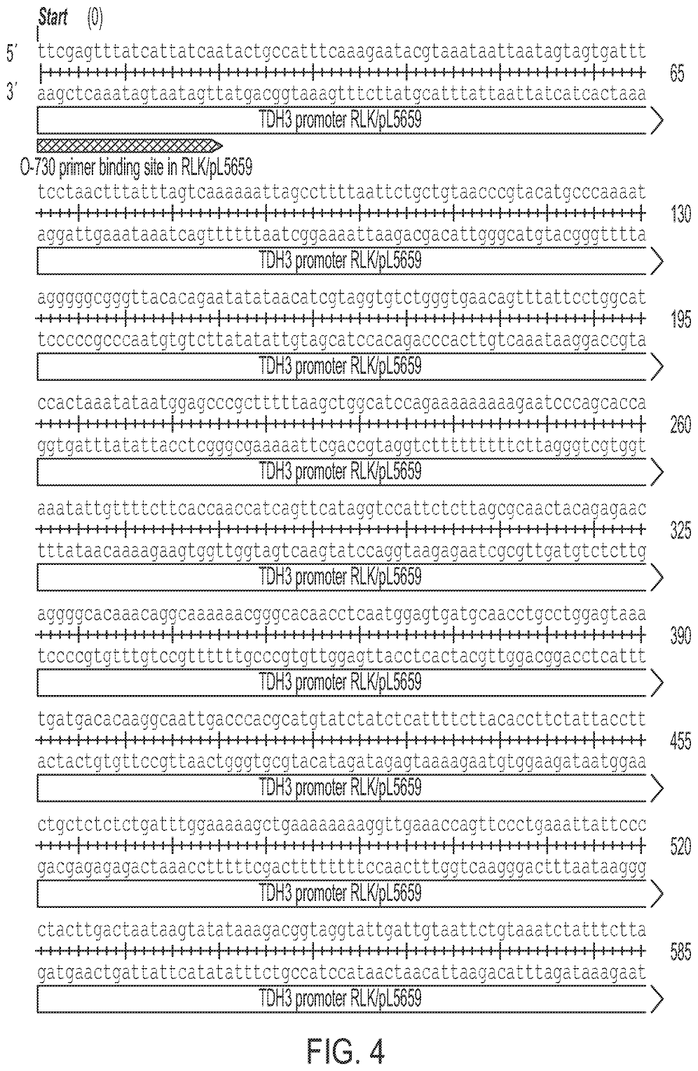

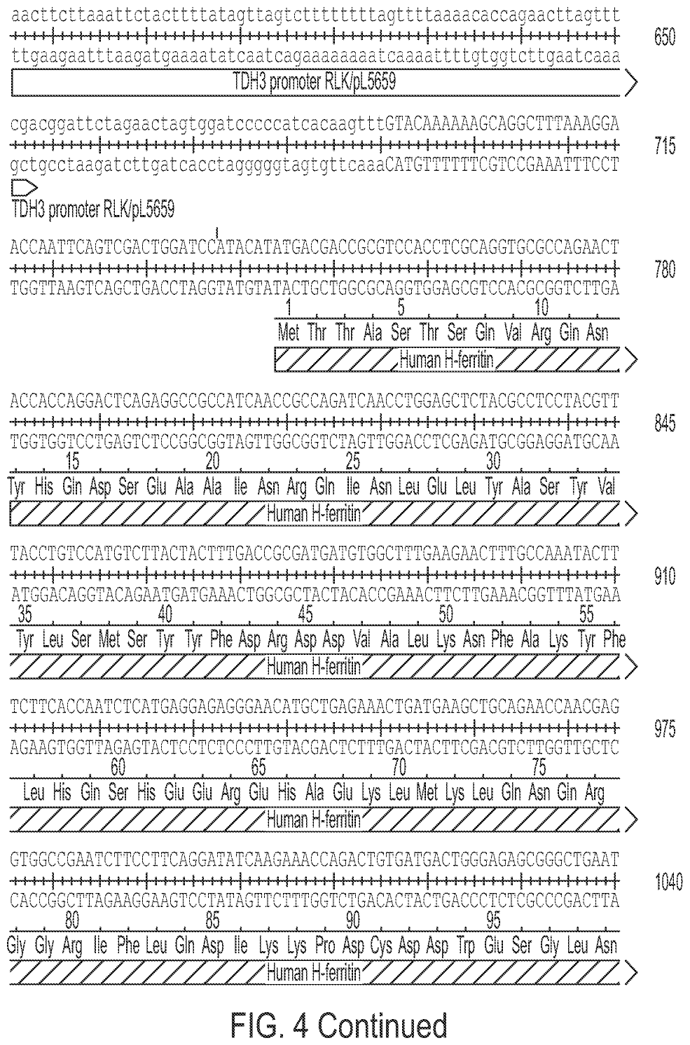

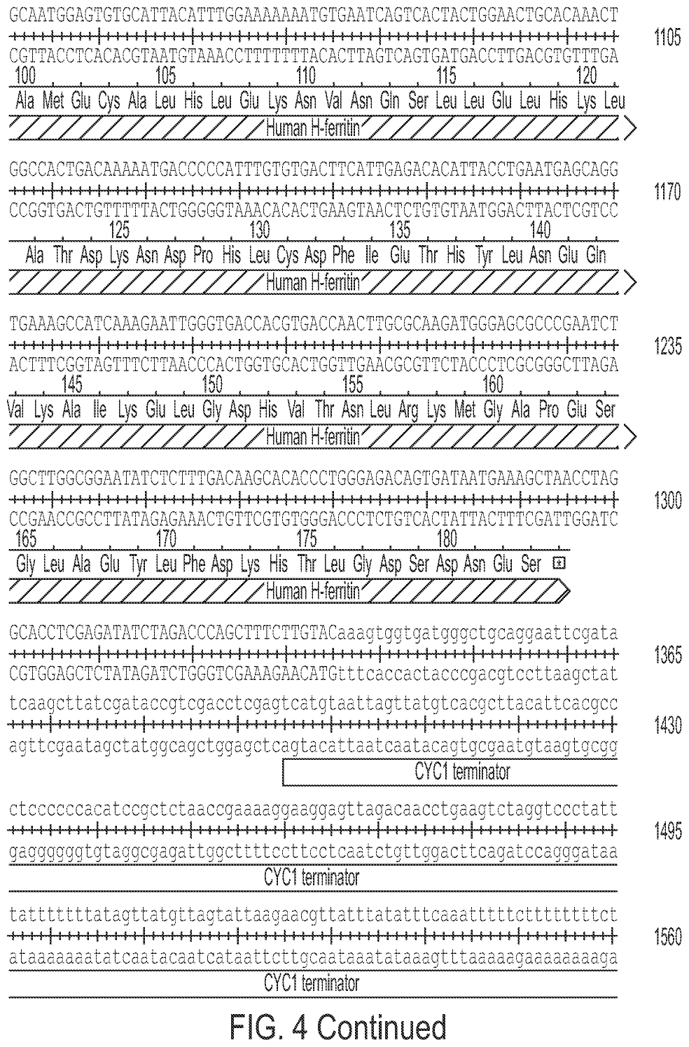

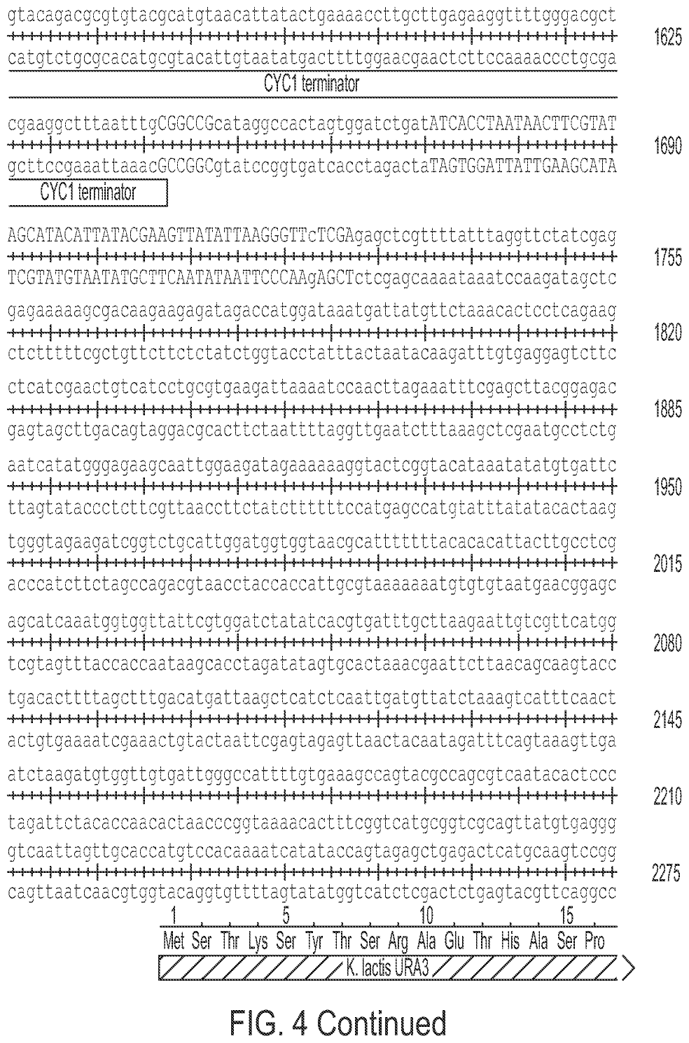

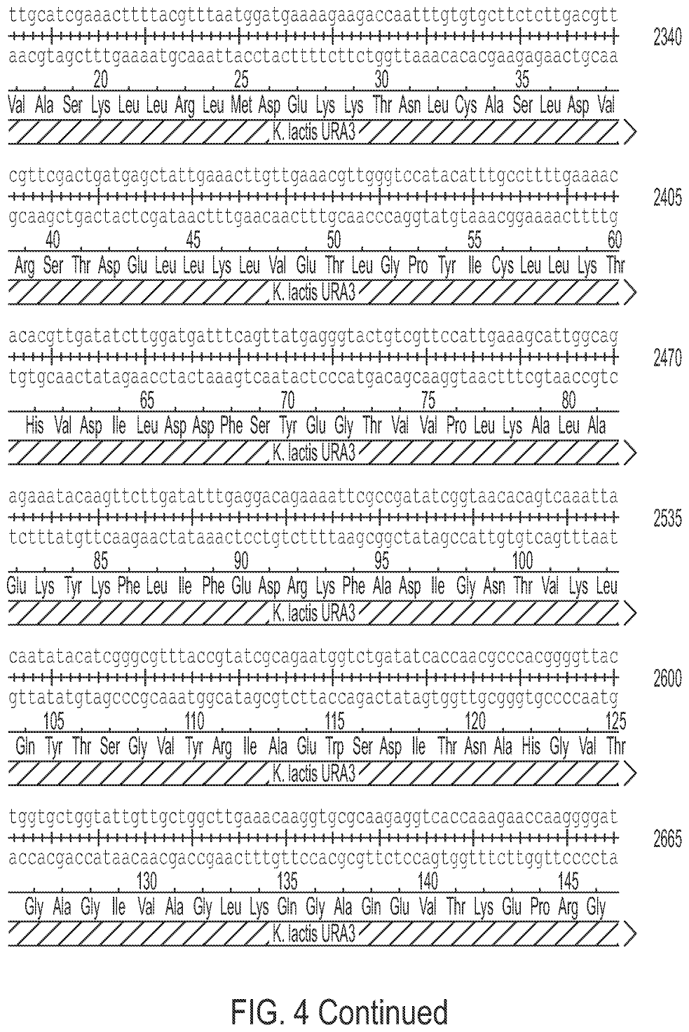

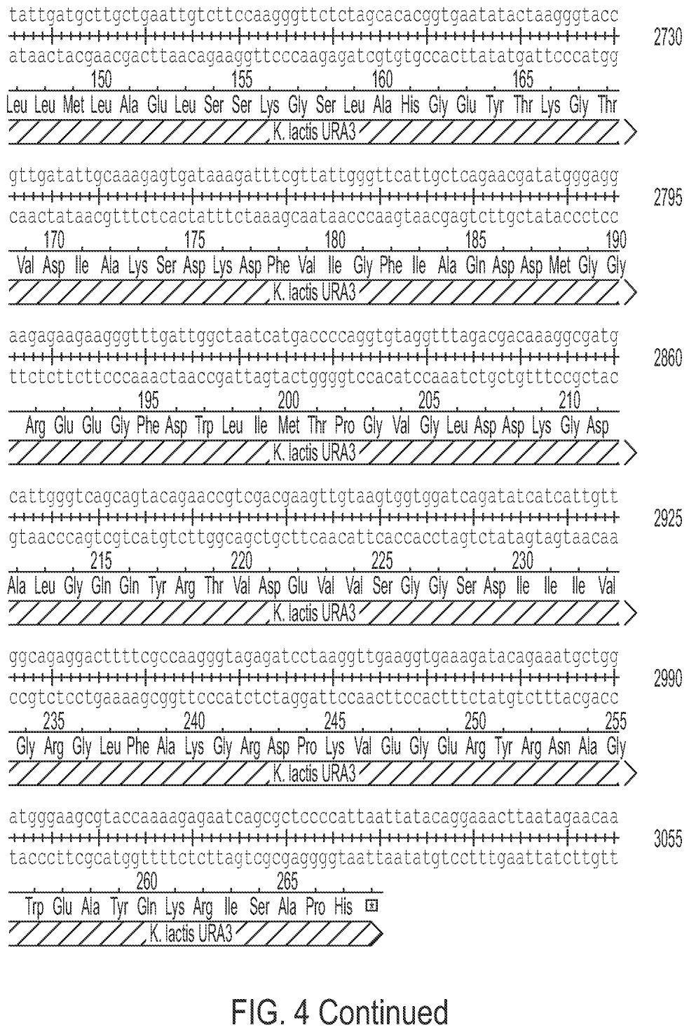

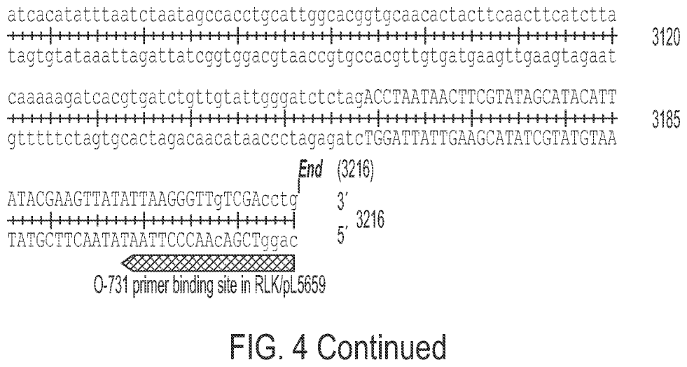

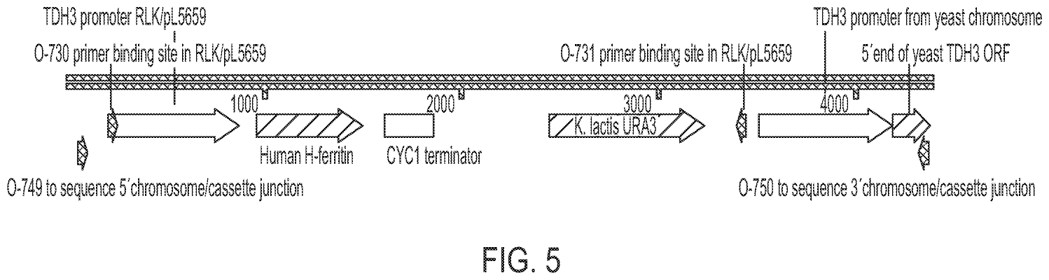

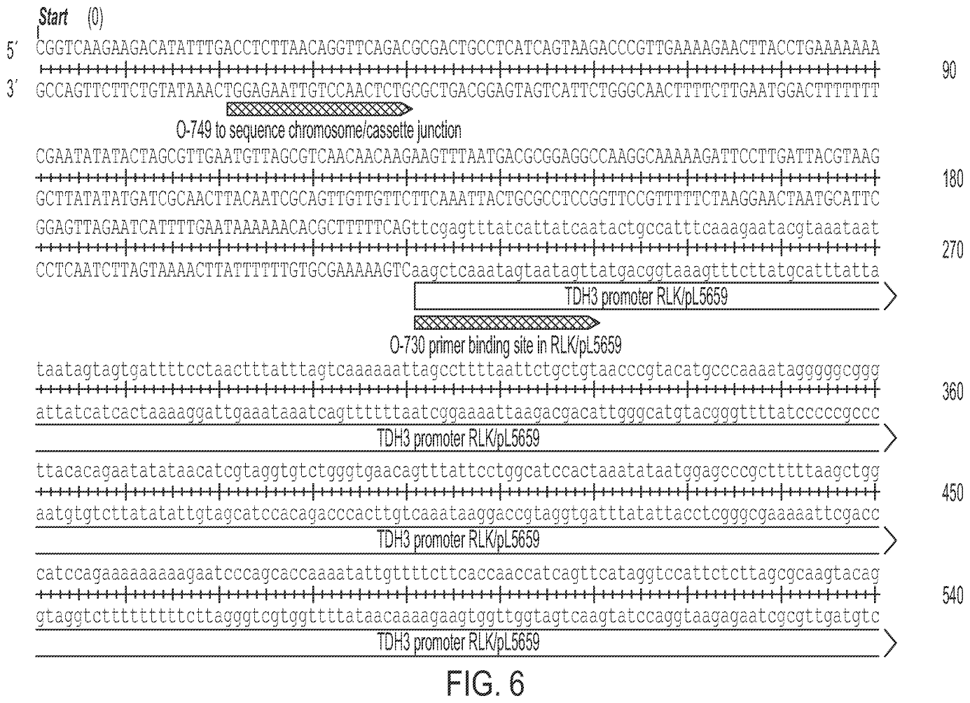

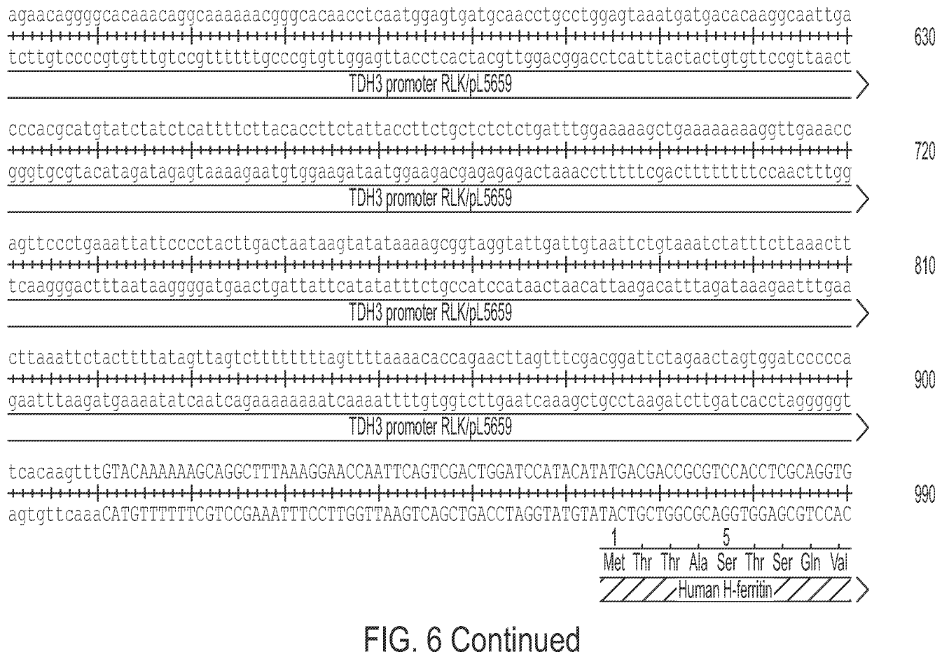

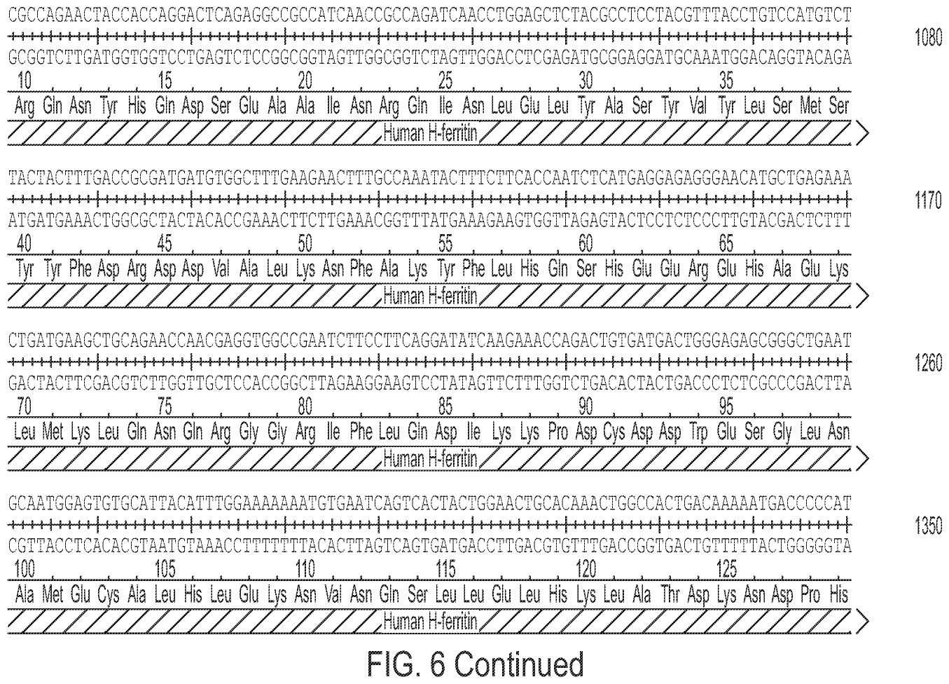

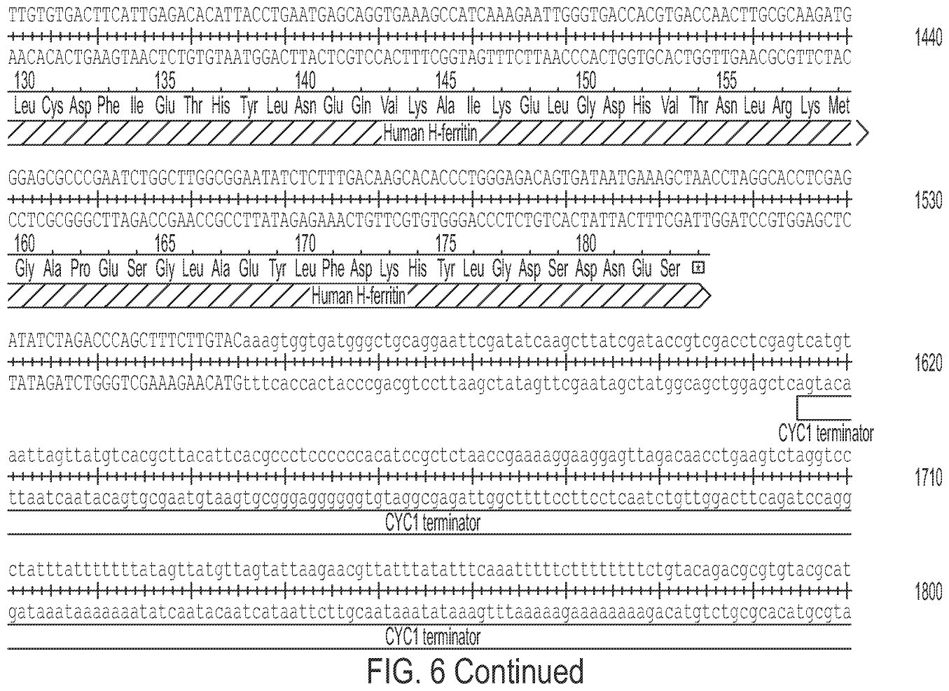

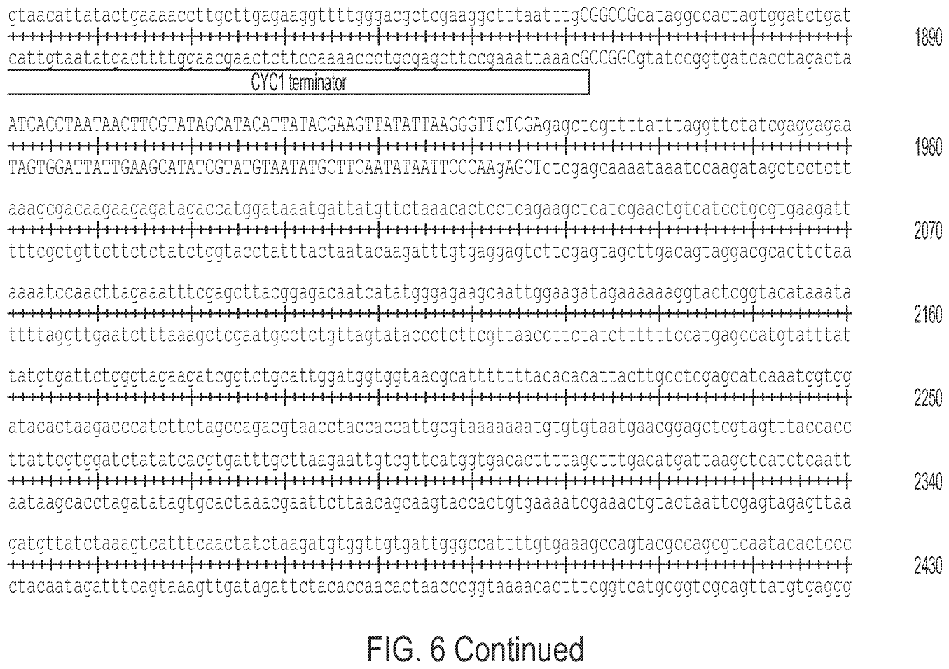

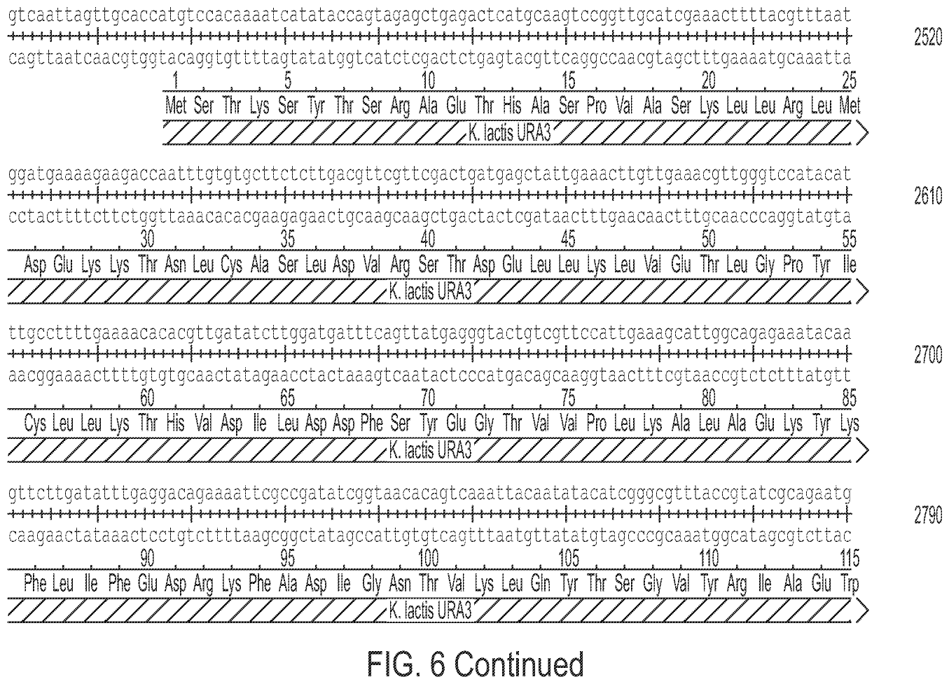

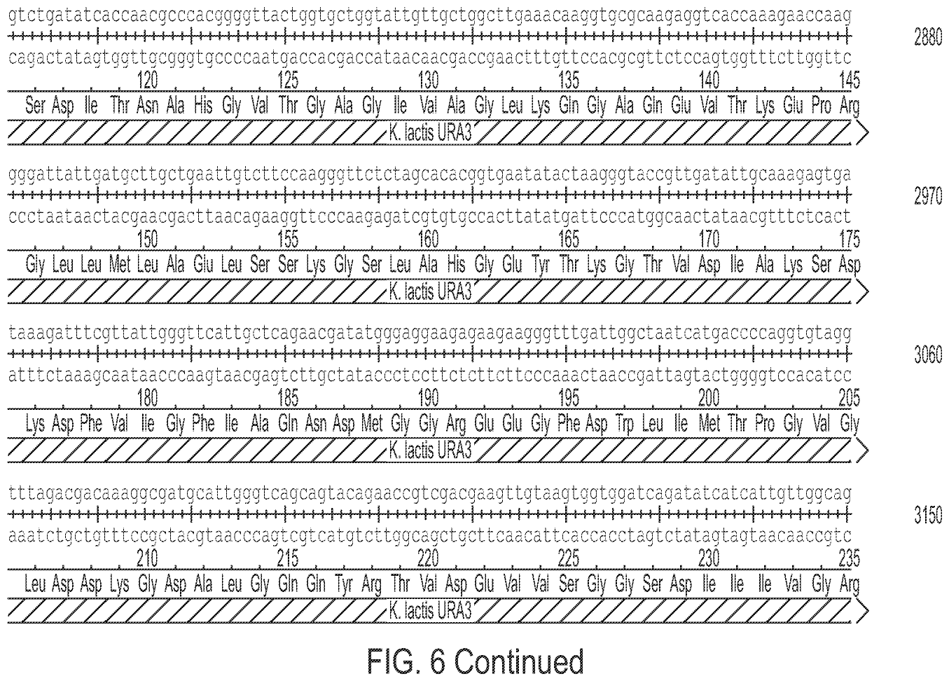

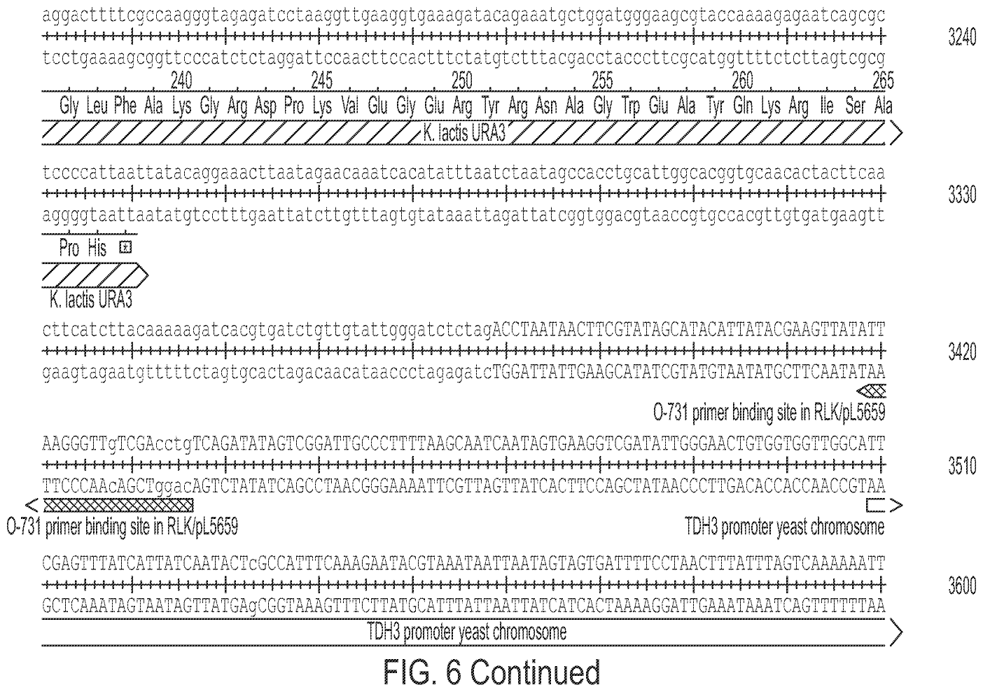

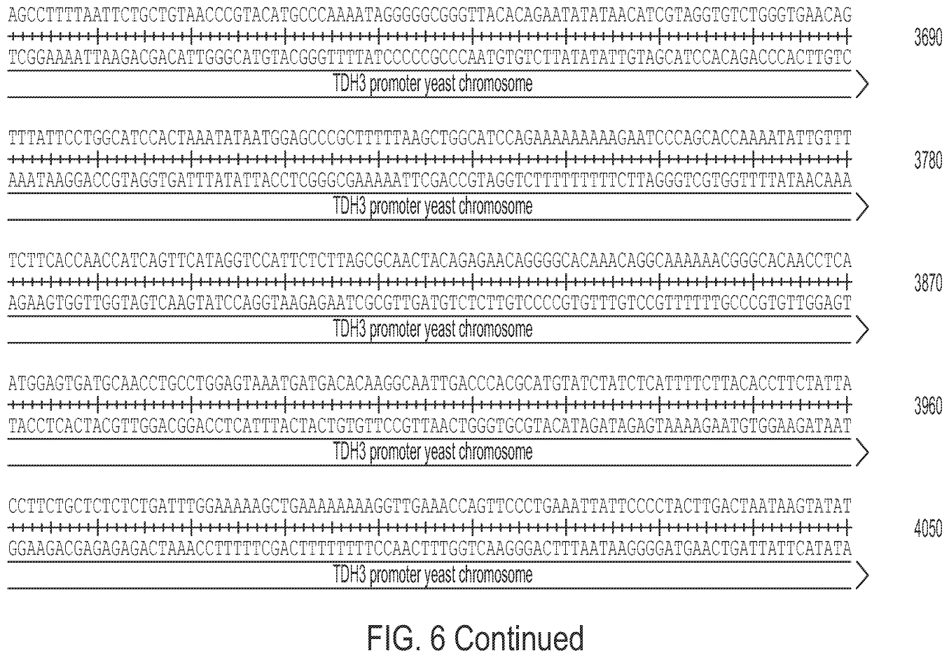

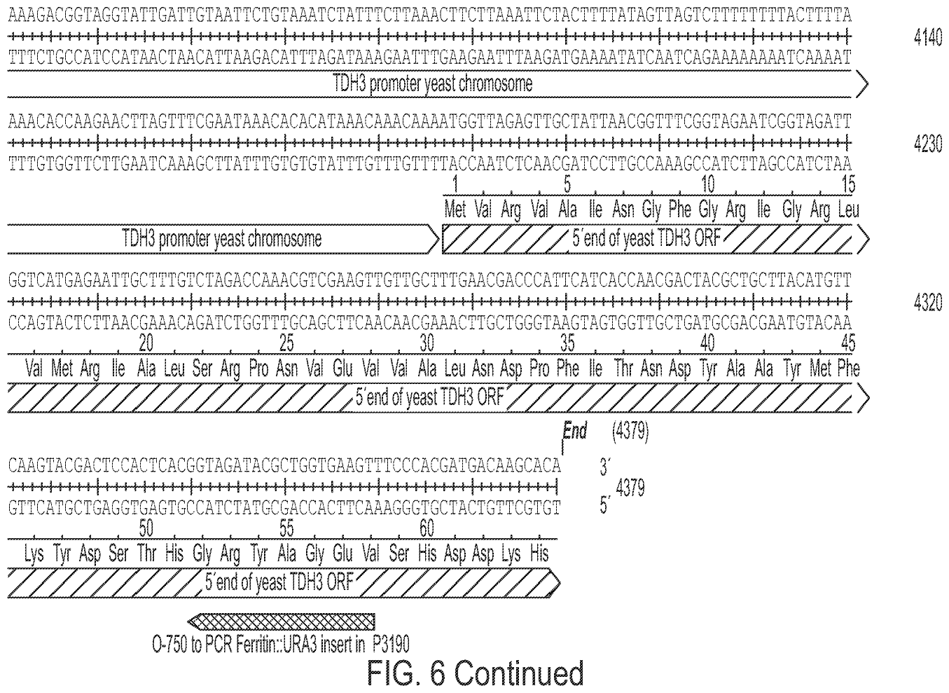

The recombinant host can be prototrophic. The prototrophic recombinant host can be haploid, diploid, polyploid, or aneuploid. As shown in FIG. 3 and FIG. 5, an H-ferritin gene may be stably integrated into the chromosome of a haploid recombinant host. FIG. 4 shows the nucleic acid sequence of the expression cassette shown in FIG. 3 (SEQ ID NO:3). FIG. 6 shows the nucleic acid sequence of the chromosomally integrated ferritin expression cassette at TDH3 (SEQ ID NO:5) shown in FIG. 5. The H-ferritin gene can be closely linked to any functional selectable marker gene, making up the genetic cassette. Suitable functional selectable marker genes will be known by those skilled in the art, for example, URA3, LEU2, TRP1, HIS3, or MET15. For example, the H-ferritin gene can be closely linked to a URA3 gene. The copy of the functional selectable marker gene that exists in the chromosome of the recombinant host can be a mutant of the functional selectable marker gene, making it non-functional.

In an example, the H-ferritin gene may be stably integrated into a chromosome of a haploid recombinant host. The H-ferritin gene can be closely linked to any functional selectable marker gene, making up the genetic cassette. Suitable functional selectable marker genes will be known by those skilled in the art, for example, URA3, LEU2, TRP1, HIS3, or MET15, or any homologues thereof. For example, the H-ferritin gene can be adjacent to a URA3 gene. The copy of the functional selectable marker gene that exists in the chromosome of the recombinant host can be a mutant of the functional selectable marker gene, making it non-functional.

In another example, a chromosome of a first haploid of a diploid, polyploid, or aneuploid recombinant microbe or host may comprise the H-ferritin gene stably integrated. In another example, both sets of chromosomes of a diploid recombinant host or at least two chromosomes of a polyploid or aneuploid may comprise the H-ferritin gene stably integrated. The H-ferritin gene can be adjacent to any functional selectable marker gene, making up the genetic cassette. Suitable functional selectable marker genes will be known by those skilled in the art, for example, URA3, LEU2, TRP1, HIS3, or MET15, or any homologues thereof. For example, the H-ferritin gene can be adjacent to a URA3 gene. The copy of the functional selectable marker gene that exists in the chromosome of the recombinant microbe or host can be a mutant of the functional selectable marker gene, making it non-functional. As will be understood by those of skill in the art, this promotes selection for the haploid comprising the genetic cassette of the first haploid and the second haploid can be a mutant URA3 gene.

The recombinant microbe or host comprising an H-ferritin gene can comprise at least one haploid cell. The recombinant microbe or host comprising an H-ferritin gene can alternatively comprise at least one diploid cell, polyploid cell, or aneuploid cell. When the recombinant host is a diploid, two haploids of opposite mating types mate to form the diploid recombinant host. One of the chromosomes in the first set of chromosomes of the recombinant diploid host can comprise the recombinant H-ferritin gene. The recombinant H-ferritin gene may be part of a chromosomally integrated genetic cassette. The recombinant H-ferritin gene encodes an amino acid sequence for the production of H-ferritin. The H-ferritin coding sequence may be placed under the control of an appropriate promoter in a genetic cassette to produce high enough levels of the iron-storage protein for the recombinant host to serve as a suitable vehicle for iron supplementation. Suitable promoters are known in the art and include promoters that induce a high level of constitutive expression and promoters whose expression can be regulated by environmental conditions. For example, in a yeast recombinant host an appropriate constitutive promoter may be the yeast TDH3 transcription promoter. For example, in a yeast recombinant host an appropriate regulatable promoter may be the yeast GAL1 promoter. In addition, the genetic constitution of the recombinant host can be further manipulated to achieve a variety of potentially advantageous outcomes. For example, proteolysis may be manipulated to enhance the stability of the iron-storage protein, or iron transport mechanisms can be manipulated to achieve desirable outcomes, such as altering the iron concentration in specific cellular compartments, including but not limited to those of the cell surface, the vacuole, or the mitochondria. For example, a transport mechanism to the vacuole may be blocked or inhibited to promote more elemental iron uptake into the ferritin. In another example, a gene could be included to promote a loading of elemental iron into ferritin. The iron content of the recombinant host may be regulated by adding known amounts of an iron compound to the medium in which the recombinant host is grown. Using the recombinant host, iron supplementation for humans and other animals can be accomplished by any of a number of means including, but not limited to, consumption or ingestion of the recombinant host. The recombinant host may be grown specifically for the purpose of iron supplementation or may be the by-product of another process (e.g., fermentation). In examples, a diploid, polyploid, or aneuploid recombinant host may only have the typical nutritional requirements of prototrophic yeast. That is, auxotrophic requirements of a set of chromosomes encoding ferritin due to genetic mutations are complemented by the presence of a non-mutant copy of the gene in a second set of chromosomes. Similarly, auxotrophic requirements of the second set of chromosomes due to genetic mutations are complemented by the presence of a non-mutant copy of the gene in the first set of chromosomes.

In examples, the second set of chromosomes may not express ferritin. The second set of chromosomes may additionally not express a functional selectable marker. That is, the second set of chromosomes may not comprise the genetic cassette. For example, the first chromosome may not comprise a functional LEU2 gene, which is involved in synthesis of leucine, an amino acid essential for the production of protein. The second set of chromosomes may comprise a functional LEU2 gene, allowing the diploid, polyploid, or aneuploid to synthesize leucine. The ability of the second set of chromosomes to synthesize leucine compensates for the inability of the first set of chromosomes to synthesize the amino acid. As a result, the diploid, polyploid, or aneuploid recombinant host comprising at least the first set of chromosomes and the second set of chromosomes is able to produce all of the normal nutritional materials prototrophic yeast synthesize (i.e., the recombinant host is a prototroph). That is, a non-mutant copy of each gene is present in at least one f the two homologous sets of chromosomes in a diploid recombinant host, at least one of three or more homologous chromosomes in a polyploid recombinant host, or at least one of the at least two sets of chromosomes in an aneuploid recombinant host. For purposes of this application, the terms “a first set of chromosomes” and “a second set of chromosomes” are used as a matter of convenience and do not indicate a particular order or limitation on the number of chromosomes (i.e., there may be two, or three, or more sets of chromosomes which are referred to herein for convenience as “the second set of chromosomes”).

For example, a first set of chromosomes of a diploid recombinant host. a polyploid recombinant host, or an aneuploid recombinant host may not comprise a functional URA3 gene, which is involved in synthesis of uracil, a nucleobase essential for the synthesis of ribonucleic acid (RNA). A second set of homologous chromosomes may comprise a functional or non-mutant URA3 gene, allowing the second set of chromosomes to synthesize uracil. The ability of the second set of chromosomes to synthesize uracil compensates for the inability of the first set of chromosomes to synthesize the nucleobase. As a result, the diploid recombinant host or polyploid recombinant host can produce all of the normal materials prototrophic yeast synthesize (i.e., the recombinant host is a prototroph). That is, at least one of the two homologous sets of chromosomes in the diploid or polyploid recombinant host contains a non-mutant copy of each gene.

The composition of the invention may optionally further comprise a second microbe or host. As used herein, the term “second” with respect to a microbe or host refers to a separate and distinct microbe or host and does not necessarily mean that only two microbes or hosts are present. The second microbe or host may comprise any of the microbes discussed above. The second microbe or host may comprise a microbe that expresses ferritin, a microbe or host that does not express ferritin, or a combination thereof. The second microbe or host may additionally or alternatively comprise a probiotic.

The composition of the present invention may optionally further comprise a prebiotic that promotes the growth of beneficial bacteria in the gut microbiome.

The composition of the invention may also include elemental iron. The elemental iron may form a ferritin-iron complex with the ferritin described above. A source of the elemental iron may be an iron salt, an organic-iron complex, an elemental iron nanoparticle, or combinations thereof. An example of a suitable iron salt includes, but is not limited to, iron sulfate. Examples of suitable organic-iron complexes include, but are not limited to, iron polymer complexes, iron carbohydrate complexes, and iron aminoglycosan complexes. These organic-iron complexes may be commercially available and/or can be synthesized by methods known in the art. Suitable non-limiting examples of iron carbohydrate complexes include iron saccharide complexes, iron oligosaccharide complexes, and iron polysaccharide complexes, such as iron carboxymaltose, iron sucrose, iron polyisomaltose (iron dextran), iron polymaltose (iron dextrin), iron gluconate, iron sorbitol, and iron hydrogenated dextran, which may be further complexed with other compounds, such as sorbitol, citric acid or gluconic acid (for example iron dextrin-sorbitol-citric acid complex and iron sucrose-gluconic acid complex), and mixtures thereof. Suitable non-limiting examples of iron aminoglycosan complexes include iron chondroitin sulfate, iron dermatan sulfate, iron keratan sulfate, each of which may be further complexed with other compounds, and mixtures thereof. Examples of iron aminoglycosan complexes include but are not limited to iron hyaluronic acid, iron protein complexes, and mixtures thereof.

The elemental iron may be present in the composition in an amount of at least one percent by weight on a dry matter basis of the microbe or host expressing ferritin and the elemental iron, such as at least five percent by weight, such as at least 5.5 percent by weight. The elemental iron may be present in the composition in an amount of no more than 15 percent by weight on a dry matter basis of the microbe or host expressing ferritin and the elemental iron, such as no more than 10 percent by weight, such as no more than eight percent by weight. The elemental iron may be present in the composition in an amount of three percent by weight to 15 percent by weight on a dry matter basis of the microbe or host expressing ferritin and the elemental iron, such as three percent by weight to 12 percent by weight, such as five percent by weight to 10 percent by weight, such as 5.5 percent by weight to 9.5 percent by weight, such as 3 percent by weight to 8 percent by weight.

The composition may include intracellular iron that is not complexed with the ferritin or a homologue thereof. The composition may also include extracellular iron (e.g., iron that is bound to a cell wall).

According to the invention, at least one percent of the elemental iron may be complexed with the ferritin, such as a least four percent, such as at least five percent, such as at least six percent, such as at least 10 percent, such as at least 15 percent, such as at least 20 percent, such as at least 30 percent, such as at least 40 percent, such as at least 50 percent, such as at least 60 percent, such as at least 75 percent of the elemental iron, and 100 percent of the elemental iron may be complexed with the ferritin, such as no more than 90 percent. According to the invention, 10 percent to 100 percent of the elemental iron may be complexed with the ferritin, such as 30 percent to 95 percent, such as 50 percent to 90 percent, such as 60 percent to 90 percent and such as 75 percent to 90 percent.

Any of the compositions described herein may be included in an ingestible form. An “ingestible form” means the composition is capable of being taken into the body orally. In examples, the microbe or host may be included in the ingestible form. For example, the ingestible form may be a medical food, a food, a food ingredient, or combinations thereof. A “medical food” is a food that has distinctive nutritional requirements that cannot be met solely by a normal diet and that is designed to be consumed or administered enterally for the treatment of a disease or disorder. In other examples, any of the compositions described herein may be in the form of a suppository. In other examples, any of the compositions described herein may be a dietary or nutritional supplement. A “dietary supplement” or “nutritional supplement” is a composition that is consumed to supplement a subject's diet in addition to meals. In other examples, any of the compositions described herein may be a pharmaceutical composition.

The compositions described herein may be in the form of a dry powder, a dispersion of the dry powder in a liquid, a suspension of the dry powder in a liquid, suppository, foam enema, liquid enema, or the like and may be formulated in such a manner as to be administered orally or rectally. The compositions of the present invention may include a pharmaceutically acceptable carrier or diluent (described herein) to form a solution, dispersion, emulsion, microemulsion, suspension, syrup, elixir or the like such that the materials may be swallowed. pH adjusters (i.e., acids, or bases) may be included to adjust pH to the appropriate level, and/or antibacterial and antifungal agents may be included to prevent the action of microorganisms. Compositions also may include formulations that control or slow release of the elemental iron in the body. In some instances, the composition may be included in a dispenser, such as a syringe, dosing vial, and the like.

Examples of ingestible diluents or carriers are sugars such as monosaccharides, disaccharides, and the like, excipients such as, but not limited to, cocoa butter and waxes; oils such as peanut oil, cottonseed oil, safflower oils, sesame oil, olive oil, corn oil, and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol; phosphate buffer solutions; other non-toxic compatible lubricants such as sodium lauryl sulfate and magnesium stearate; coloring agents, releasing agents, coating agents, preservatives and antioxidants according to the judgment of the formulator.

The compositions described herein may be expelled from a pressurized container, or may be in the form of powders, granules, or lozenges. Examples of suitable binders and fillers include, but are not limited to, magnesium stearate, microcrystalline cellulose, cellulose gel, cellulose gum, carboxymethyl cellulose, wood pulp, soy lecithin, glycine, monosodium glutamate, vegetable protein, seaweed or extract, carrageenan, or combinations thereof.

As used herein, the term “pharmaceutically acceptable” means acceptable for use in the pharmaceutical and veterinary arts, compatible with other ingredients of the formulation, and not toxic or otherwise unacceptable commensurate with a reasonable benefit/risk ratio. As used herein, “pharmaceutically acceptable carrier” includes any and all solvents, diluents, or other liquid vehicle, dispersion or suspension aids, surface active agents, isotonic agents, thickening and emulsifying agents, stabilizers, preservatives, solid binders, lubricants, and the like, as suited to the particular dosage form desired. Remington's Pharmaceutical Sciences ed. by Gennaro, Mack Publishing, Easton, PA 1995 provides various carriers used in formulating pharmaceutical compositions and known techniques for the preparation thereof. Such carriers may also be used, where appropriate, in formulating nutritional or dietary supplements, food ingredients, foods and medical foods.

For subjects diagnosed with an iron deficiency disorder, a skilled artisan understands that various factors influence the dosage required to treat such a subject effectively, and that accordingly a dosage and administration may be chosen by a medical professional (e.g., an attending physician) in view of the subject to be treated and may be adjusted for sufficient levels of the active agent(s) or to maintain the desired effect. Additional factors that may be taken into account include the severity of the disease state, e.g., intermediate or advanced stage of disease; age, weight, gender and overall health of the subject; diet, time and frequency of administration; form of iron deficiency; route of administration; drug combinations; reaction sensitivities; prior treatments; and tolerance/response to therapy. The composition described herein may be administered, for example, every 30 minutes, hourly or daily; multiple times per day; weekly, multiple times per week; bi-weekly; monthly; and the like.

The composition described herein may be used to provide a source of elemental iron and/or ferritin to a subject. The composition may be administered to a subject not diagnosed with an iron deficiency or to a subject diagnosed with an iron deficiency.

The Recommended Dictary Allowance (RDA) for elemental iron according to the Food and Nutrition Board (FNB) at the Institute of Medicine (IOM) of the National Academies administered orally is 8 milligrams of iron per day for a male subject between 19 and 50 years of age not diagnosed with an iron deficiency disorder and 18 milligrams per day for a female subject similarly not diagnosed with an iron deficiency disorder. The tolerable daily upper intake level (UL) for elemental iron from food and supplements based on the amounts of iron that are associated with adverse gastrointestinal effects following supplemental intakes of iron salts established by the FNB is 45 milligrams for male and female subjects 19 years of age or older not diagnosed an iron deficiency disorder (e.g., 0.56 milligrams/kilogram/day for an 80-kilogram subject). Medical professionals sometimes prescribe intakes higher than the UL for subjects diagnosed with an iron deficiency with one standard of care daily recommendation for iron salts such as ferrous sulfate being 165 milligrams to 195 milligrams per day (2.1 milligrams/kilogram/day to 2.5 milligrams/kilogram/day for an 80-kilogram subject). It has surprisingly been found that a dosage of a composition comprising, consisting essentially of or consisting of a microbe or host expressing ferritin and elemental iron can be administered orally in excess of a dosage of elemental iron recommended by a nutritional authority for a subject not diagnosed with an iron deficiency disorder as well as in excess of a dosage of elemental iron recommended by a medical professional (e.g., a treating physician) for a subject diagnosed with an iron deficiency disorder without an adverse effect, including without gastrointestinal distress or intolerance. This allows treating a subject with greater than the UL to address the subject's iron deficiency (where previously treating only up to the UL did not). Representative administration of a composition comprising, consisting essentially of or consisting of a microbe or host expressing ferritin and elemental iron can be orally administered in amounts of 5 to 100 times the UL for elemental iron (e.g., 2.8 milligrams/kilograms/day to 56.2 milligrams/kilograms/day for an 80-kilogram subject) to male or female subjects not diagnosed with an iron deficiency or diagnosed with an iron deficiency without an adverse effect, such as but not limited to 10 to 100 times the UL for elemental iron (e.g., 5.6 milligrams/kilograms/day to 56.2 milligrams/kilograms/day for an 80-kilogram subject), such as 10 to 90 times the UL for elemental iron (e.g., 5.6 milligrams/kilograms/day to 50.6 milligrams/kilograms/day for an 80-kilogram subject), such as 15 to 80 times the UL for elemental iron (e.g., 8.4 milligrams/kilograms/day to 45 milligrams/kilograms/day for an 80-kilogram subject), such as 20 to 70 times the UL for elemental iron (11.2 milligrams/kilograms/day to 39.4 milligrams/kilograms/day for an 80-kilogram subject), such as 25 to 60 times the UL for elemental iron (14.1 milligrams/kilograms/day to 33.8 milligrams/kilograms/day for an 80-kilogram subject) and such as 30 to 50 times the UL for elemental iron (16.9 milligrams/kg/day to 28.1 milligrams/kilograms/day for an 80-kilogram subject).

Representative administration of a composition comprising, consisting essentially of or consisting of a microbe or host expressing ferritin and elemental iron can be administered in amounts of 5 milligrams/kilograms/day elemental iron to 65 milligrams/kilograms/day, such as but not limited to 5 milligrams/kilograms/day to 50 milligrams/kilograms/day, 7.5 milligrams/kilograms/day to 45 milligrams/kilograms/day, 10 milligrams/kilograms/day to 40 milligrams/kilograms/day, 15 milligrams/kilograms/day to 35 milligrams/kilograms/day and 20 milligrams/kilograms/day to 30 milligrams/kilograms/day.

A representative elemental iron calculation is:

$$UL Fe for an adult male = 45 mg / day$$

$$10 \times UL = 450 mg / day$$

$$Adult male estimated weight = 80 kg$$

$$Daily Fe administration for 80 kg male at 10 \times UL = 450 / 80 = 5.6 mg / kg / day .$$

TABLE 1
Daily Iron Administration Comparison for
Adults with and without Iron Deficiency

| | | | | Ferritin |
|---|---|---|---|---|
| | | | Fe | Complex |
| | RDA | UL | Deficient | Composition |
| Subject | (mg/kg/day) | (mg/kg/day) | (mg/kg/day) | (mg/kg/day) |
| 80 kg male | 0.1 | 0.56 | 2.1-2.4 | 2.8-56.2 |
| 60 kg female | 0.3 | 0.75 | 2.8-3.2 | 3.75-75 |

As described herein, the dosage level may be administered as a single dose administered to the subject or patient, or through multiple administrations (multiple doses) that achieve, for example, a daily dosage level over the course of a day. The dosage level may also be a total amount of iron administered multiple times per week, weekly, bi-weekly, or monthly administration divided by the number of days between administration, wherein the dose administers elemental iron in a dosage level described above on a per day average. Because the amount of iron provided in a composition comprising, consisting essentially of or consisting of a microbe or host expressing ferritin and elemental iron can be administered orally in amounts greatly exceeding the UL (e.g., 5 to 100 times the UL) without adverse effect, the composition provides a tolerable composition that can be taken by subjects desiring or needing iron supplementation, including otherwise healthy pregnant women that might require 27 milligrams of iron per day (e.g., 0.34 mg/kg/day for an 80 kg subject) or more. The oral administration of the composition can also meet or exceed doses (e.g., daily dosages) that have heretofore potentially only been given intravenously.

In addition to administration, e.g., daily administration, of elemental iron in excess of a dosage of elemental iron recommended by a nutritional authority for a subject not diagnosed with an iron deficiency disorder as well as in excess of a dosage of elemental iron recommended by a medical professional (e.g., a treating physician) for a subject diagnosed with an iron deficiency disorder, it has also surprisingly been found that the amount of ferritin administered therewith in a composition comprising, consisting essentially of or consisting of (a) a microbe or host expressing ferritin and (b) elemental iron does not produce any adverse effect. A microbe or host of yeast of the various species of the genus Saccharomyces contains approximately 40 percent protein by dry weight. Ferritin expressed in such yeast represents approximately one percent to 15 percent of that protein by dry weight, such as 3 percent, such as 4 percent, such as 5 percent, such as 6 percent, such as 7 percent, such as 8 percent or such as 10 percent of the total protein. A composition of a microbe or host of yeast expressing ferritin and elemental iron administered by or to a subject at 5 to 100 times the UL for elemental iron would provide the subject with approximately 2.5 milligrams/kilograms/day to 50 milligrams/kilograms/day ferritin for an 80-kilogram subject. It is estimated that newborns and breast-feeding babies receive approximately 0.3 milligrams/kilograms/day to 0.5 milligrams/kilograms/day. The following representative examples use a FerritinFeComplex containing 4.5 percent iron by dry weight (gram/gram) and a yeast microbe or host containing 40 percent protein by dry weight with 10 percent of that protein being ferritin.

### 80-Kilogram Subject:

Fc: 2.8 milligrams/kilograms/day to 56.2 milligrams/kilograms/day

Ferritin: 2.8 mg Fc/kg/day×80 kg×FerritinFcComplex/0.045 Fe×0.04 Ferritin/FerritinFeComplex=199 mg Ferritin/day (2.5 mg Ferritin/kg/day)

Ferritin: 56.2 mg Fe/kg/day×80 kg×FerritinFeComplex/0.045 Fc×0.04 Ferritin/FerritinFeComplex=3996 mg Ferritin/day (50 mg Ferritin/kg/day)

60-kilogram Subject:

Fc: 3.75 milligrams/kilograms/day to 75 milligrams/kilograms/day

Ferritin: 3.75 mg Fc/kg/day×60 kg×FerritinFcComplex/0.045 Fe×0.04 Ferritin/FerritinFcComplex=200 mg Ferritin/day (3.3 mg Ferritin/kg/day)

Ferritin: 75 mg Fe/kg/day×60 kg×FerritinFcComplex/0.045 Fc×0.04 Ferritin/FerritinFcComplex=4000 mg Ferritin/day (66.7 mg Ferritin/kg/day)

The composition may be administered as a single dose or as multiple doses (i.e., first, second, third, etc. doses) administered contemporancously or sequentially, such that administration of a first dose of the composition is followed by administration of a second dose of the composition, or vice versa. When the first and second doses are administered sequentially, the method may comprise waiting a period of time between the administration of the doses. First, second, third, etc. doses may comprise the same or different amounts of elemental iron. As used herein, the term “sequentially” refers to a treatment protocol in which administration of a first dose of a composition of the present invention follows administration of a second dose of a composition of the present invention. As used herein, the term “contemporaneously” refers to administration of a first dose of a composition of the present invention and administration of a second dose of a composition of the present invention, wherein the first and second doses are separate and are administered at substantially the same time.

According to the present invention, a subject not diagnosed with an iron deficiency disorder or a subject that is or has been diagnosed with an iron deficiency disorder may be treated by administering to the subject any of the compositions described herein, such as administering a therapeutically effective amount of any of the compositions described herein. The administering of the composition to the subject may be done by the subject himself/herself or another subject (e.g., a medical professional, a caretaker, a family member). The composition may be provided to the subject or the administrator for the subject along with instructions for administration of the composition (e.g., written instructions on a label of a container containing the composition).

The composition of the invention may be administered to manage or regulate iron levels in a subject either to provide iron (e.g., elemental iron) by administering a composition including a microbe or host expressing ferritin and elemental iron or to promote or assist in the processing or excretion of iron stored in the body of the subject by administering to a subject a composition comprising, consisting essentially of or consisting of a microbe or host expressing ferritin in the absence or substantial absence of elemental iron. A dosage of ferritin in the composition may be 50 milligrams/day or more, such as 100 milligrams/day, such as 200 milligrams/day, such as 300 milligrams/day or more, such as 200 milligrams/day to 4000 milligrams/day. The administering of the composition comprising, consisting essentially of or consisting of a microbe or host expressing ferritin in the absence or substantial absence of elemental iron to the subject may be done by the subject himself/herself or another subject (e.g., a medical professional, a caretaker, a family member). A composition may representatively be formulated in a manner described above without intentionally adding elemental iron to the microbe or host expressing ferritin. It is appreciated that some amount of iron may be present in the microbe or host expressing ferritin (e.g., a yeast may contain some amount of iron). The composition may be provided to the subject or the administrator for the subject along with instructions for administration of the composition (e.g., written instructions on a label of a container containing the composition). Such promotion or assistance in the processing or excretion of iron by a composition including a microbe or host expressing ferritin in the absence of elemental iron may, for example, be beneficial to subjects that may present (e.g., be diagnosed with) or be at risk for an iron overload disorder, including, but not limited to, subjects with hereditary hemochromatosis, secondary hemochromatosis or juvenile hemochromatosis.

The compositions of the invention may be administered for a purpose of altering a composition of a gut bacterial microbiome in a subject. For example, a method of altering the composition of the gut bacterial microbiome in a subject may comprise, consist essentially of or consist of administering to the subject a composition comprising, consisting essentially of or consisting of (a) a microbe or host expressing ferritin and (b) elemental iron, wherein the amount of elemental iron in the composition administered (1) to a subject not diagnosed with an iron deficiency disorder can exceed a dosage (e.g., a daily dosage) of elemental iron recommended by a nutritional authority or (2) to a subject diagnosed with an iron deficiency disorder can exceed a dosage (e.g., a daily dosage) of elemental iron recommended by a medical professional without an adverse effect. In another example, a method of altering the composition of the gut bacterial microbiome in a subject may comprise, consist essentially of or consist of administering to a subject a composition comprising, consisting essentially of or consisting of a microbe or host expressing ferritin, with the proviso that when a daily dosage of ferritin in the composition is less than 200 milligrams/day, the composition is free of elemental iron.

The method of altering the composition of the gut bacterial microbiome in a subject may comprise modulating the relative abundance of Gammaproteobacteria class bacteria and/or Clostridia bacteria in a subject's gut. The Gammaproteobacteria class bacteria comprise bacteria from the Enterobacteriales order, Enterobacteriaceae family, and/or Klebsiella genus. The Clostridia class bacteria comprise bacteria from the Clostridium, Anaerosporobacter, and/or Pygmaiobacter genera. In one microbiome study, adult human subjects consumed a composition comprising (a) yeast expressing ferritin and (b) 5.7 percent by weight elemental iron (171 mg) at 1.5 grams/day for two weeks followed by 3 grams/day for six weeks. The study showed that a relativeabundance of Gammaproteobacteria class bacteria and/or Clostridia class bacteria may be reduced by at least −2 as determined by linear discriminant analysis.

The method of altering the composition of the gut bacterial microbiome in a subject may comprise modulating the relative abundance of Erysipelotrichia class bacteria and/or Clostridia class bacteria in a subject's gut. The Erysipelotrichia class bacteria comprise bacteria from the Erysipelotrichales order, Erysipelotrichaceae family, Candidatus genus, and/or Stoquefichus species. The Clostridia class bacteria comprise bacteria from the Ruminococcaceae family. In the microbiome study referenced above, the relative abundance of Erysipelotrichia class bacteria and/or Clostridia class bacteria may be increased at least +2 as determined by linear discriminant analysis.

The method of altering the composition of the gut bacterial microbiome in a subject may comprise reducing biofilm formation (biofilm forming bacteria) in a gut of a subject. Gut biofilm is associated with numerous inflammatory disorders including Clostridioides difficile and Irritable Bowel Disease. The microbiome study referenced above saw statistically significant reductions in negative (pathogenic) biofilm formation (p=0.023; Linear Discriminant Analysis >2.0). The reduction in biofilm formation may be as determined by PICRETSt analysis.

The method of altering the composition of the gut bacterial microbiome in a subject may comprise reducing the bacterial chemotaxis in a gut of a subject. The microbiome study referenced above saw a reduction of bacterial chemotaxis at week 2 which became statistically significant at weck 4 (p=0.04) and weck 8 (p=0.034). A trend upward was seen at weck 10, two weeks after the consumption of the composition (yeast expressing ferritin and elemental iron) was stopped. The reduction in bacterial chemotaxis may be as determined by PICRETSt analysis.

The lack of gastrointestinal side effects of the compositions of the invention with or without elemental iron dosages in excess of that recommended by a Nutritional Authority or, in the case of a subject diagnosed with an iron deficiency disorder, in excess of that recommended by a medical professional may be due to the surprisingly positive effect treatment with the composition of the present invention according to the methods of the present invention has on a microbiome of the subject. Surprising results were demonstrated by evaluating a microbiome both before, during and after treatment. It has been surprisingly discovered that treatment does not negatively impact the bacterial species richness of the patient or subject's microbiome while increasing the relative abundance of species of genus of bacteria that do not negatively impact the gut microbiome (e.g., Candidatus, Erysipelotrichales, Erysipelotrichia, Erysipelotrichaceae, and/or Ruminococcaceae) while reducing the relative abundance of species of genus of bacteria that do negatively impact the gut microbiome (e.g., Klebsiella, Enterobacteriales, Enter obacteriaceae, Clostridium, Anaerosporobacter, and/or Pygmaiobacter). Accordingly, treatment with the composition of the present invention according to the method of the present invention surprisingly improves the overall gut microbiome of a subject, in addition to improving quantitative and qualitative measures of the iron supplementation, the iron deficiency disorder or the iron processing or excretion of iron stored in the body of the subject, as the case may be. For example, according to the invention, such improvements may result in at least 1 week, such as at least 2 weeks, such as at least 4 weeks, such as at least 6 weeks, such as at least 8 weeks, such as at least 10 weeks, such as at least 12 weeks, following the administering of any of the compositions described herein.

In the foregoing specification, the invention has been described with reference to specific exemplary embodiments thereof. It will be evident that various modifications may be made thereto without departing from the broader spirit and scope of the invention as set forth in the following claims. The specification and drawings are, accordingly, to be regarded in an illustrative sense rather than a restrictive sense.

### EXAMPLES

### Example 1. Preparation of a Microbe or Host Expressing Ferritin

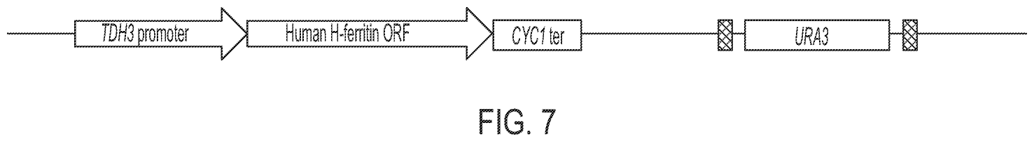

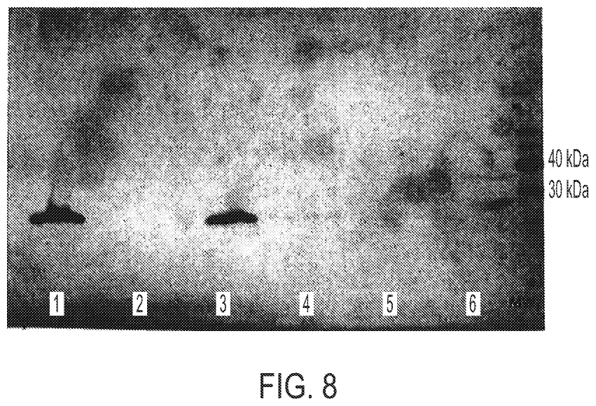

An H-ferritin expression cassette for S. cerevisiae, shown in FIG. 7, expressing human H-ferritin under the control of the yeast constitutive TDH3 transcriptional promoter was generated by PCR from plasmid RLK/pL5659, which was derived from pAG426GPD-ccdB (AddGene, Cambridge, Mass.), by inserting the H-ferritin coding sequence and the URA3 gene from Kluyveromyces lactis. The PCR product was transformed into the yeast strain BY4741 and allowed to integrate into the yeast chromosome using standard methods, for example, as described in Hinnen et al., PNAS USA 75: 1929-1933, 1978. Yeast transformants containing the expression cassette were recovered via the selectable URA3 marker, and lysates of transformed yeast were prepared in 50 mM Tris-HCl (pH 7.4), 150 mM NaCl using glass beads. Twenty-five micrograms (μg) of total protein, determined by the DC Protein Assay (Bio-Rad), were fractionated by SDS-PAGE and transferred to a nitrocellulose filter. The blot was probed with H-ferritin polyclonal antibody diluted 1:2000 (Covance PA 1192). The secondary antibody was anti-rabbit IgG diluted 1:5000 (GE Amersham) and signal was detected using Western Lightning-ECL (Perkin Elmer). The results, shown in FIG. 8, indicate that the amount of expression of recombinant H-ferritin depends on the site of chromosomal integration. Recombinant strain RLK3190, containing a chromosomally integrated H-ferritin expression cassette, unexpectedly expressed dramatically higher levels of human H-ferritin compared with other chromosomal sites of integration. This level matches or exceeds the amount of H-ferritin produced by strain RLK3177, which contains an extra-chromosomal plasmid bearing multiple copies of the H-ferritin expression cassette. In RLK3190, the expression cassette integrated at the chromosomal location of TDH3 by homologous recombination based on the homology between the TDH3 promoter on the expression cassette and the chromosomal gene. This cassette and others can readily be engineered for insertion at this site using standard techniques in the art.

The expression cassette in the RLK3190 strain constitutively produces human H-ferritin at high levels when it is integrated into the TDH3 locus on the yeast chromosome.

### Example 2. Preparation of a Composition Comprising a Microbe or Host Expressing Ferritin and Elemental Iron (Yeast Ferritin Complex)

The RLK3190 strain from Example 1 was grown in a yeast fermentation medium supplemented with iron (6 mM FeSO₄). Following fermentative growth, the Yeast Ferritin Complex was concentrated and dried to less than 10 percent moisture. The Yeast Ferritin Complex was then assessed for iron and ferritin levels.

### Example 3. Toxicity Study of Composition Comprising a Microbe or Host Expressing Ferritin and Elemental Iron in Sprague Dawley Rats

In an example studying the toxicity of a composition comprising a microbe expressing ferritin and elemental iron in rats, the Yeast Ferritin Complex of Example 2 was formed into a powder that contained 4.5 percent elemental iron by weight on a dry matter basis. 12 male and 12 female Sprague Dawley rats were randomly assigned to two dose groups (Groups 1 through 2), 6 rats/sex/group. Formulations of a test article, prepared in accordance with the Example 2 (the Yeast Ferritin Complex or YFC), or a control article, Reverse Osmosis (RO) deionized water, were administered by once daily oral gavage from Days 1 to 42 of this study to these male and female rats at the dose levels shown in Table 2. The dose volume was adjusted daily on the basis of the mostly recently recorded body weight. The rat subjects had a weight on the order of 226 to 250 grams (males) and 176 to 200 grams (females).

TABLE 2
Dosage Level

| | | | Dose | Dose | No. of | |
|---|---|---|---|---|---|---|
| Group | | Dose Level | Volume | Concentration | Animals | |
| No. | Test Material | (mg/kg/day) | (mL/kg) | (mg/mL) | Males | Females |
| 1 | Control Article | 0 | 10 | 0 | 6 | 6 |
| 2 | YFC | 1000 | 10 | 100 | 6 | 6 |

Study parameters evaluated throughout the 42-day study included: viability, clinical observations, body weights, food consumption, clinical pathology (hematology, coagulation, clinical chemistry-iron panel, ferritin, urinalysis, feces analysis), gross necropsy observations, organ weights, and histopathology evaluation.

### Results

### Mortality

All rats survived to scheduled euthanasia on day 43. There were no test-article-related unscheduled deaths in this study.

### Clinical and Necropsy Observations

There were no Yeast Ferritin Complex-related clinical signs observed in male and female rats at a dose of 1000 mg/kg/day. An isolated incidence of fur loss was noted in one rat in the control group from day 24 to day 26. This fur loss resolved and was not noted after day 26. There were no Yeast Ferritin Complex-related gross changes at necropsy.

### Body Weights

There were no Yeast Ferritin Complex-related effects on mean body weights in male and female rats at 1000 mg/kg/day. Mean body weights were comparable in male and female rats in the control group and the Yeast Ferritin Complex-administered group.

### Food Consumption

There were no Yeast Ferritin Complex-related effects on food consumption values throughout the overall duration of the study from DS 1 to DS 42. Food consumption values were comparable among the three dose groups for the weekly intervals.

### Iron Panel

1000 mg/kg/day Yeast Ferritin Complex having 4.5 percent elemental iron represents 45 mg/kg/day elemental iron which amounts to 9 mg/day for a 200-gram rat and 11.2 mg/day for a 250-gram rat. The individual values for iron panel analysis [Total iron, Unsaturated Iron Binding Capacity (UIBC) and Total Iron Binding Capacity (TIBC)] have been appended to this report for review. Mildly decreased iron in females and mildly increased iron in males with associated changes in UIBC (decrease for females and increase for males) was noted but these changes are of uncertain relationship to the test article given the small magnitude that is considered to be within biologic variability and the fact that the direction of change is the opposite for males and females.

### Ferritin Levels

There were no clear Yeast Ferritin Complex-related effects on ferritin levels in male or female rats at a dose of 1000 mg/kg/day. 1000 mg/kg/day represents approximately 40 mg/kg/day ferritin (e.g., H-ferritin) which amounts to approximately 8 mg/day for a 200-gram rat or 10 mg/day for a 250-gram rat.

1000 mg YFC/kg/day×0.4 protein/YFC×0.1 FTH1/protein×1 kg/1000 g×200 g rat=8 mg FTHI/day

### SUMMARY

Administration of Yeast Ferritin Complex by once daily oral gavage for 42 days was well tolerated in rats at levels of 1000 mg/kg/day. No adverse effects related to clinical observations, body weights and gross pathology was observed.

### Example 4. Comparison Study of Haploid Yeast Ferritin Complex and Diploid Yeast Ferritin Complex in Sprague Dawley Rats

Twenty-day old male Sprague Dawley rats (Envigo) were housed 1 per cage in hanging wire cages in a temperature (23±2° C.) and humidity (40%) controlled room maintained on a 12:12 hr light/dark cycle (lights on 0600 to 1800). Rats were fed an iron deficient diet (“ID diet”) of 2 ppm iron or supplement diets ad libitum as indicated. The ID diet was prepared following the recipe of the American Institute of Nutrition (AlN)-93G diet with cornstarch as the sole source of carbohydrate and without the addition of iron. Iron levels for all diets were verified using atomic absorption spectrometry after wet digestion with nitric acid (Perkin Elmer). All experimental protocols were in accordance with The National Institutes of Health Animal Care guidelines and were approved by The Pennsylvania State University Institutional Animal Care and Use Committee.

Rats were fed an ID diet for 26 days (P21-P47), which produced anemia (mean hematocrit and hemoglobin levels were 5.3±0.2 and 16.2±0.1%, respectively). At P47, rats were then divided into 2 dietary groups (n=3-5/group) balanced by body weight. Dietary group 1 was fed a diet comprising a haploid Yeast Ferritin Complex (53 μg iron/g diet; n=4). The haploid Yeast Ferritin Complex comprises a single chromosome that comprises the chromosomally integrated ferritin expression cassette in FIG. 5. The haploid Yeast Ferritin Complex is auxotrophic. Dietary group 2 was fed a diet comprising a diploid Yeast Ferritin Complex as described in the application (49 μg iron/g diet; n=3). The diploid Yeast Ferritin Complex comprises two chromosomes—a first chromosome comprises the chromosomally integrated ferritin expression cassette in FIG. 5 and a second chromosome is absent the chromosomally integrated ferritin expression cassette. The diploid Yeast Ferritin Complex is prototrophic. Rats were fed the assigned diet for 14 days, and food consumption was monitored throughout the study.

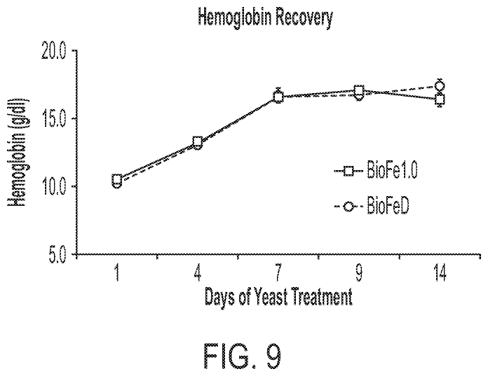

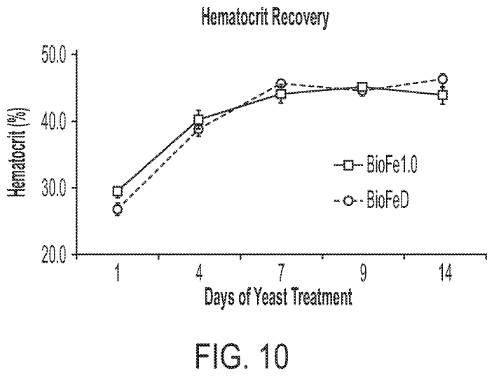

The results shown in FIG. 9 and FIG. 10 show that the diploid Yeast Ferritin Complex provided comparable hemoglobin recovery and hematocrit recovery, respectively, as the haploid Yeast Ferritin Complex.

### Example 5. Comparison Study of Haploid Yeast Ferritin Complex and Diploid Yeast Ferritin Complex

Preparation of Haploid S. cerevisiae Yeast

Haploid S. cerevisiae was grown in synthetic complete (SC) medium lacking uracil with 6 mM FeSO₄ at 30° C. with shaking for two days. Cells were collected by centrifugation and washed once with sterile water. Following resuspension, cells were incubated at 65° C. for 30 min. to pasteurize. Cells were then washed two additional times with sterile water to remove unincorporated iron and lyophilized.

Preparation of Diploid S. cevervisiae Yeast

Diploid S. cerevisiae was grown in synthetic complete (SC) medium lacking uracil with 6 mM FeSO₄ at 30° C. with shaking for two days. Cells were collected by centrifugation and washed once with sterile water. Following resuspension, cells were incubated at 65° C. for 30 min. to pasteurize. Cells were then washed two additional times with sterile water to remove unincorporated iron and lyophilized.

### Analysis of Haploid and Diploid Yeast Samples

To analyze the haploid and diploid yeast samples for ferritin, an aliquot of the yeast preparation was viewed with electron microscopy. To further demonstrate the presence of FHT1, a western blot analysis was performed on the yeast lysate. The samples were run on a 4-20% Tri-Glycine SDS gel. Following this, it was transferred to a PVDF membrane and probed first with an antibody against H-ferritin (1:500, Cell signaling) followed by a secondary antibody (1:5000, GE healthcare). To determine if the ferritin in the yeast contained iron, a 25 μl of the yeast lysate was run on a 4-20% Tris-Glycine SDS gel (BioRad) and used to assess iron using Perls' strain. Briefly, the gel was rinsed once with de-ionized water followed by incubation with a solution consisting of two parts potassium ferricyanide (Sigman Aldrich, cat #702587) and one part 10% HCl solution for 30 min. The gel was subsequently washed with deionized water several times to remove background and observed for a blue Perls' reaction.

In order to analyze the iron content of the yeast samples, yeast was digested in nitic acid and the iron amounts were determined using atomic absorption spectrophotometry.

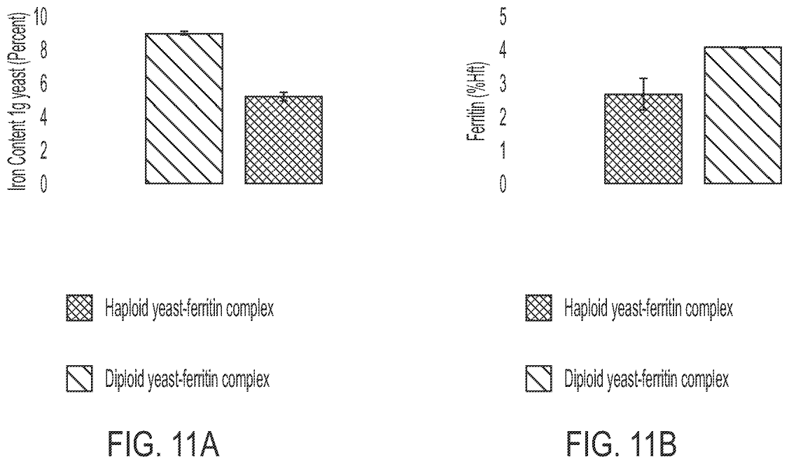

The results shown in FIG. 11A compared the iron content of the haploid yeast sample and the diploid yeast sample based on weight percent of a 1 g sample of yeast. As shown, the diploid yeast sample had higher iron levels (9%) compared to the haploid yeast sample (5%). The results shown in FIG. 11B compared the H-ferritin content of the haploid yeast sample and the diploid yeast sample. As shown, the haploid yeast sample had a greater amount of H-ferritin (4.12%) compared to the diploid yeast sample (2.76%). Thus, despite the fact there was less H-ferritin in the diploid yeast sample, there was more iron as compared to the haploid yeast sample. Additionally, despite the differences, the haploid yeast and the diploid yeast performed equally well in in vivo studies. This suggests that the diploid yeast was more efficient at absorbing iron, while still maintaining performance.

We claim:

1. A method comprising administering to a subject a composition comprising (a) a microbe or host expressing ferritin and (b) elemental iron, wherein the amount of elemental iron in the composition administered (1) to a subject not diagnosed with an iron deficiency disorder can exceed a dosage of elemental iron recommended by a nutritional authority or (2) to a subject diagnosed with an iron deficiency disorder can exceed a dosage of elemental iron recommended by a medical professional without an adverse effect.

2. (canceled)

3. The method of claim 1, wherein a daily dosage of elemental iron recommended by a national nutritional authority is 8 milligrams for an adult male subject and/or 18 milligrams for an adult female subject.

4. (canceled)

5. The method of claim 1, wherein a daily dosage of elemental iron recommended is greater than 45 milligrams for a subject not diagnosed with an iron deficiency disorder or greater than 195 milligrams for a subject diagnosed with an iron deficiency disorder.

6. The method of claim 1, wherein a daily dosage of elemental iron is 5 mg/kg/day to 50 mg/kg/day.

7-10. (canceled)

11. The method of claim 1, wherein a dosage of ferritin in the composition is 200 milligrams/day to 4000 milligrams/day.

12. The method of any of claim 1, wherein the microbe or host comprises a yeast.

13-14. (canceled)

15. A method comprising administering to a subject a composition comprising (a) a microbe or host expressing ferritin and (b) elemental iron, wherein the amount of elemental iron in the composition administered (1) to a subject not diagnosed with an iron deficiency disorder is in excess of a dosage of elemental iron recommended by a nutritional authority or (2) to a subject diagnosed with an iron deficiency disorder is in excess of a dosage of elemental iron recommended by a medical professional.

16. (canceled)

17. The method of claim 15, wherein a daily dosage of elemental iron recommended by a nutritional authority is 8 milligrams for an adult male subject and/or 18 milligrams for an adult female subject.

18. (canceled)

19. The method of claim 15, wherein a daily dosage of elemental iron recommended is greater than 45 milligrams for a subject not diagnosed with an iron deficiency disorder or greater than 195 milligrams for a subject diagnosed with an iron deficiency disorder.

20. The method of claim 15, wherein e a daily dosage of elemental iron is 5 mg/kg/day to 50 mg/kg/day.

21-23. (canceled)

24. The method of claim 15, wherein a dosage of ferritin in the composition is 200 milligrams/day to 4000 milligrams/day.

25. The method of claim 15, wherein the microbe or host comprises a yeast.

26-27. (canceled)

28. A method comprising:
providing a composition comprising (a) a microbe or host expressing ferritin and (b) elemental iron; and
providing instructions for administration of the composition on a dosage basis, wherein the amount of elemental iron in a dosage of the composition (1) to a subject not diagnosed with an iron deficiency disorder exceeds a dosage of elemental iron recommended by a nutritional authority or (2) to a subject diagnosed with an iron deficiency disorder exceeds a dosage of elemental iron recommended by a medical professional.

29. (canceled)

30. The method of claim 28, wherein a daily dosage of elemental iron recommended by a nutritional authority is 8 milligrams for an adult male subject and/or 18 milligrams for an adult female subject.

31. (canceled)

32. The method of claim 28, wherein a daily dosage of elemental iron recommended is greater than 45 milligrams for a subject not diagnosed with an iron deficiency disorder or greater than 195 milligrams for a subject diagnosed with an iron deficiency disorder.

33. The method of claim 28, wherein a daily dosage of elemental iron is 5 mg/kg/day to 50 mg/kg/day.

34-35. (canceled)

36. A method comprising administering to a subject a composition comprising a microbe or host expressing ferritin, with the proviso that when a daily dosage of ferritin in the composition is less than 200 milligrams/day, the composition is free of elemental iron.

37. The method of claim 36, wherein a dosage of ferritin in the composition is 200 milligrams/day to 4000 milligrams/day.

38. The method of claim 36, wherein the composition comprises elemental iron.

39. (canceled)

40. The method of claim 36, wherein the microbe or host comprises a yeast.

41-44. (canceled)

45. A method of altering a composition of a gut bacterial microbiome in a subject comprising administering to the subject a composition comprising a microbe or host expressing ferritin, with the proviso that when a daily dosage of ferritin in the composition is less than 200 milligrams/day, the composition is free of elemental iron.

46. The method of claim 45, wherein a dosage of ferritin in the composition is 200 milligrams/day to 4000 milligrams/day.

47. The method of claim 45, wherein the composition comprises elemental iron.

48. (canceled)

49. The method of claim 45, wherein the microbe or host comprises a yeast.

50-51. (canceled)
